SEQUENCE LISTING (81 sequences)

SEQ ID NO: 1 - DNA, 1660 bp, Streptococcus agalactiae
atgaaattatcgaagaagttattgttttcggctgctgttttaacaatggtggcggggtcaactgttgaaccagtagctcagtttgcgactggaatgagtattgtaagagctgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaatttgaaattactgataaatttgcagatggcttgacttataaatctgttggaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcaatccctaatactggtggtattggtacggctatctttgtcgctatcggtgctgcggtgatggcttttgctgttaaggggatgaagcgtcgtacaaaagataac

SEQ ID NO: 2 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 3 - protein, 5 aa, Streptococcus agalactiae
IPNTG

SEQ ID NO: 4 - protein, 484 aa, Streptococcus agalactiae
MAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 5 - protein, 270 aa, Streptococcus agalactiae
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNWTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 6 - protein, 212 aa, Streptococcus agalactiae
MTLVKNQDALDKATANTDDAAFLEIPVASTINEKAVLGKAIENTFELQYDGTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETIAPEGYVIPDKEIEFTVSQTSYNTKPTDITVDSADATPDTIKNNKRPS

SEQ ID NO: 7 - protein, 81 aa, Streptococcus agalactiae
DGEVISNYAKLGDNVKGLQGVQFKRYKVKTDISVDELKKLTTVEAADAKVGTILEEGVSLPQKTNAQGLVVDALDSKSNVR

SEQ ID NO: 8 - protein, 65 aa, Streptococcus agalactiae
GLQGVQFKRYKVKTDISVDELKKLTTVEAADAKVGTILEEGVSLPQKTNAQGLVVDALDSKSNVR

SEQ ID NO: 9 - protein, 30 aa, Streptococcus agalactiae
YDGTPDKADNPKPSNPPRKPEVHTGGKRFV

SEQ ID NO: 10 - protein, 9 aa, Streptococcus agalactiae
NPKPSNPPR

SEQ ID NO: 11 - protein, 13 aa, Streptococcus agalactiae
DALDSKSNVRYLY

SEQ ID NO: 12 - protein, 13 aa, Streptococcus agalactiae
SNVRYLYVEDLKN

SEQ ID NO: 13 - protein, 13 aa, Streptococcus agalactiae
DAAFLEIPVASTI

SEQ ID NO: 14 - protein, 13 aa, Streptococcus agalactiae
FLEIPVASTINEK

SEQ ID NO: 15 - protein, 13 aa, Streptococcus agalactiae
IPVASTINEKAVL

SEQ ID NO: 16 - protein, 13 aa, Streptococcus agalactiae
ASTINEKAVLGKA

SEQ ID NO: 17 - protein, 13 aa, Streptococcus agalactiae
INEKAVLGKAIEN

SEQ ID NO: 18 - protein, 13 aa, Streptococcus agalactiae
KAVLGKAIENTFE

SEQ ID NO: 19 - protein, 13 aa, Streptococcus agalactiae
LGKAIENTFELQY

SEQ ID NO: 20 - protein, 13 aa, Streptococcus agalactiae
AIENTFELQYDHT

SEQ ID NO: 21 - protein, 13 aa, Streptococcus agalactiae
NTFELQYDHTPDK

SEQ ID NO: 22 - protein, 13 aa, Streptococcus agalactiae
ELQYDHTPDKADN

SEQ ID NO: 23 - protein, 13 aa, Streptococcus agalactiae
YDHTPDKADNPKP

SEQ ID NO: 24 - protein, 13 aa, Streptococcus agalactiae
TPDKADNPKPSNP

SEQ ID NO: 25 - protein, 13 aa, Streptococcus agalactiae
KADNPKPSNPPRK

SEQ ID NO: 26 - protein, 13 aa, Streptococcus agalactiae
NPKPSNPPRKPEV

SEQ ID NO: 27 - protein, 13 aa, Streptococcus agalactiae
PSNPPRKPEVHTG

SEQ ID NO: 28 - protein, 13 aa, Streptococcus agalactiae
PPRKPEVHTGGKR

SEQ ID NO: 29 - protein, 13 aa, Streptococcus agalactiae
KPEVHTGGKRFVK

SEQ ID NO: 30 - protein, 13 aa, Streptococcus agalactiae
VHTGGKRFVKKDS

SEQ ID NO: 31 - protein, 13 aa, Streptococcus agalactiae
GGKRFVKKDSTET

SEQ ID NO: 32 - protein, 13 aa, Streptococcus agalactiae
RFVKKDSTETQTL

SEQ ID NO: 33 - protein, 13 aa, Streptococcus agalactiae
KKDSTETQTLGGA

SEQ ID NO: 34 - protein, 13 aa, Streptococcus agalactiae
STETQTLGGAEFD

SEQ ID NO: 35 - protein, 13 aa, Streptococcus agalactiae
TQTLGGAEFDLLA

SEQ ID NO: 36 - protein, 13 aa, Streptococcus agalactiae
LGGAEFDLLASDG

SEQ ID NO: 37 - protein, 13 aa, Streptococcus agalactiae
AEFDLLASDGTAV

SEQ ID NO: 38 - protein, 13 aa, Streptococcus agalactiae
DLLASDGTAVKWT

SEQ ID NO: 39 - protein, 13 aa, Streptococcus agalactiae
LASDGTAVKWTDA

SEQ ID NO: 40 - protein, 13 aa, Streptococcus agalactiae
MAEVSQERPAKTT

SEQ ID NO: 41 - protein, 13 aa, Streptococcus agalactiae
VSQERPAKTTVNI

SEQ ID NO: 42 - protein, 13 aa, Streptococcus agalactiae
ERPAKTTVNIYKL

SEQ ID NO: 43 - protein, 13 aa, Streptococcus agalactiae
AKTTVNIYKLQAD

SEQ ID NO: 44 - protein, 13 aa, Streptococcus agalactiae
TVNIYKLQADSYK

SEQ ID NO: 45 - protein, 13 aa, Streptococcus agalactiae
IYKLQADSYKSEI

SEQ ID NO: 46 - protein, 13 aa, Streptococcus agalactiae
LQADSYKSEITSN

SEQ ID NO: 47 - protein, 13 aa, Streptococcus agalactiae
DSYKSEITSNGGI

SEQ ID NO: 48 - protein, 13 aa, Streptococcus agalactiae
KSEITSNGGIENK

SEQ ID NO: 49 - protein, 13 aa, Streptococcus agalactiae
ITSNGGIENKDGE

SEQ ID NO: 50 - protein, 13 aa, Streptococcus agalactiae
NGGIENKDGEVIS

SEQ ID NO: 51 - protein, 13 aa, Streptococcus agalactiae
IENKDGEVISNYA

SEQ ID NO: 52 - protein, 13 aa, Streptococcus agalactiae
KDGEVISNYAKLG

SEQ ID NO: 53 - protein, 13 aa, Streptococcus agalactiae
EVISNYAKLGDNV

SEQ ID NO: 54 - protein, 13 aa, Streptococcus agalactiae
SNYAKLGDNVKGL

SEQ ID NO: 55 - protein, 13 aa, Streptococcus agalactiae
AKLGDNVKGLQGV

SEQ ID NO: 56 - protein, 13 aa, Streptococcus agalactiae
GDNVKGLQGVQFK

SEQ ID NO: 57 - protein, 13 aa, Streptococcus agalactiae
VKGLQGVQFKRYK

SEQ ID NO: 58 - protein, 13 aa, Streptococcus agalactiae
LQGVQFKRYKVKT

SEQ ID NO: 59 - protein, 13 aa, Streptococcus agalactiae
VQFKRYKVKTDIS

SEQ ID NO: 60 - protein, 13 aa, Streptococcus agalactiae
KRYKVKTDISVDE

SEQ ID NO: 61 - protein, 13 aa, Streptococcus agalactiae
KVKTDISVDELKK

SEQ ID NO: 62 - protein, 13 aa, Streptococcus agalactiae
TDISVDELKKLTT

SEQ ID NO: 63 - protein, 13 aa, Streptococcus agalactiae
SVDELKKLTTVEA

SEQ ID NO: 64 - protein, 13 aa, Streptococcus agalactiae
ELKKLTTVEAADA

SEQ ID NO: 65 - protein, 13 aa, Streptococcus agalactiae
KLTTVEAADAKVG

SEQ ID NO: 66 - protein, 13 aa, Streptococcus agalactiae
TVEAADAKVGTIL

SEQ ID NO: 67 - protein, 13 aa, Streptococcus agalactiae
AADAKVGTILEEG

SEQ ID NO: 68 - protein, 13 aa, Streptococcus agalactiae
AKVGTILEEGVSL

SEQ ID NO: 69 - protein, 13 aa, Streptococcus agalactiae
GTILEEGVSLPQK

SEQ ID NO: 70 - protein, 13 aa, Streptococcus agalactiae
LEEGVSLPQKTNA

SEQ ID NO: 71 - protein, 13 aa, Streptococcus agalactiae
GVSLPQKTNAQGL

SEQ ID NO: 72 - protein, 13 aa, Streptococcus agalactiae
LPQKTNAQGLVVD

SEQ ID NO: 73 - protein, 13 aa, Streptococcus agalactiae
KTNAQGLVVDALD

SEQ ID NO: 74 - protein, 13 aa, Streptococcus agalactiae
AQGLVVDALDSKS

SEQ ID NO: 75 - protein, 13 aa, Streptococcus agalactiae
LVVDALDSKSNVR

SEQ ID NO: 76 - protein, 13 aa, Streptococcus agalactiae
DSKSNVRYLYVED

SEQ ID NO: 77 - protein, 13 aa, Streptococcus agalactiae
RYLYVEDLKNSPS

SEQ ID NO: 78 - protein, 13 aa, Streptococcus agalactiae
YVEDLKNSPSNIT

SEQ ID NO: 79 - protein, 13 aa, Streptococcus agalactiae
DLKNSPSNITKAY

SEQ ID NO: 80 - protein, 13 aa, Streptococcus agalactiae
NSPSNITKAYAVP

SEQ ID NO: 81 - protein, 13 aa, Streptococcus agalactiae
SPSNITKAYAVPF

(19) United States
(12) Patent Application Publication
(10) Pub. No.: US 2012/0141521 A1
(43) Pub. Date: Jun. 7, 2012

(54) Immunogenic Compositions for Streptococcus agalactiae

(76) Inventors: Domenico Maione, Siena (IT); Guido Grandi, Siena (IT); Nathalie Norais, Siena (IT)

(21) Appl. No.: 13/339,628

(22) Filed: Dec. 29, 2011

Related U.S. Application Data
(63) Continuation of application No. 12/303,999, filed on Dec. 9, 2008, now abandoned.
(60) Provisional application No. 60/812,145, filed on Jun. 9, 2006.

(51) Int. Cl. A61K 39/09; A61P 37/04; A61P 31/04; C07K 14/315

(52) U.S. Cl. 424/190.1; 530/350

(57) ABSTRACT

The invention relates to immunogenic polypeptides derived from epitopes in a Streptococcus agalactiae (“GBS”) protein GBS 80 and their use as prophylactic, diagnostic and therapeutic compositions. The invention also provides nucleic acids encoding the immunogenic polypeptides. Also provided are vectors useful for making such immunogenic polypeptides and host cells transformed with such vectors. In particular, the invention relates to a group immunogenic polypeptides derived from GBS 80. The compositions may include one or more of the immunogenic polypeptides either alone or with other antigenic components. For example, the immunogenic polypeptides may be combined with other GBS antigens to provide therapeutic compositions with broader range. In addition, the immunogenic polypeptides may also include flanking portions of the GBS 80 protein

This application incorporates by reference a 29.7 kb text file created on Dec. 29, 2011 and named “PAT051702_sequencelisting.txt,” which is the sequence listing for this application.

### FIELD OF THE INVENTION

The invention relates to immunogenic polypeptides derived from a Streptococcus agalactiae (“GBS”) protein GBS 80 and their use as diagnostic, prophylactic, and therapeutic compositions. In particular, the invention relates to a group of immunogenic polypeptides derived from GBS 80. The compositions may include one or more of the immunogenic polypeptides either alone or with other immunogenic components. For example, the immunogenic polypeptides may be combined with other GBS antigens to provide therapeutic compositions with broader range. In addition, the immunogenic polypeptides may also include flanking portions of the GBS 80 protein.

### BACKGROUND OF THE INVENTION

GBS has emerged in the last 20 years as the major cause of neonatal sepsis and meningitis that affect 0.5-3 per 1000 live births, and an important cause of morbidity among the older age group affecting 5-8 per 100,000 of the population. Current disease management strategies rely on intrapartum antibiotics and neonatal monitoring which have reduced neonatal case mortality from >50% in the 1970's to less than 10% in the 1990's. Nevertheless, there is still considerable morbidity and mortality and the management is expensive. 15-35% of pregnant women are asymptomatic carriers and at high risk of transmitting the disease to their babies. Risk of neonatal infection is associated with low serotype specific maternal antibodies and high titers are believed to be protective. In addition, invasive GBS disease is increasingly recognized in elderly adults with underlying disease such as diabetes and cancer.

The “B” in “GBS” refers to the Lancefield classification, which is based on the antigenicity of a carbohydrate which is soluble in dilute acid and called the C carbohydrate. Lancefield identified 13 types of C carbohydrate, designated A to O, that could be serologically differentiated; the organisms that most commonly infect humans are found in groups A, B, D, and G. Within group B, strains can be divided into at least 9 serotypes (Ia, Ib, Ia/c, II, III, IV, V, VI, VII and VIII) based on the structure of their polysaccharide capsule. In the past, serotypes Ia, Ib, II, and III were equally prevalent in normal vaginal carriage and early onset sepsis in newborns. Type V GBS has emerged as an important cause of GBS infection in the USA, however, and strains of types VI and VIII have become prevalent among Japanese women.

The genome sequence of a serotype V strain 2603 V/R has been published (Ref. 1) and various polypeptides for use a vaccine antigens have been identified (Ref. 2). The vaccines currently in clinical trials, however, are based on polysaccharide antigens. These suffer from serotype specificity and poor immunogenicity, and so there is a need for effective vaccines against S. agalactiae infection.

It is an object of the invention to provide improved compositions for providing immunity against, and treatment of, GBS disease and/or infection. The compositions are based on a group of immunogenic polypeptides derived from GBS 80.

### SUMMARY OF THE INVENTION

Applicants have discovered that an immunogenic GBS antigen, GBS 80, is particularly suitable for immunization purposes, which may be used in combination with other GBS antigens. Applicants have identified four regions within GBS 80 that are of particular interest given their demonstrated antigenic qualities.

One aspect of the present invention provides an immunogenic composition comprising an immunogenic polypeptide from GBS 80 or a fragment thereof, wherein said immunogenic polypeptide is a fragment of GBS 80 that includes one of the regions identified in this application (especially SEQ ID NO:7-12 or antigenic fragment thereof) and may include additional portions of GBS 80. The length of the fragment may vary depending on the amino acid sequence of the specific immunogenic polypeptide, but the fragment is preferably at least 7 consecutive amino acids, (e.g. 8, 10, 12, 14, 16, 18, 20, 25, 30, 35, 40, 50, 60, 70, 80, 90, 100, 150, 200 or more).

The immunogenic polypeptides may include polypeptide sequences having sequence identity to the identified immunogenic polypeptides (especially SEQ ID NO:7-12 or antigenic fragments thereof). The degree of sequence identity may vary depending on the amino acid sequence in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more). Polypeptides having sequence identity include homologs, orthologs, allelic variants and functional mutants of the identified GBS 80 immunogenic polypeptides. The immunogenic polypeptides may include polypeptide sequences encoded by nucleic acid sequences that hybridize under high stringency wash conditions (see below for representative conditions) to nucleic acids encoding an identified immunogenic polypeptide (especially SEQ ID NO: 7-12 or antigenic fragments thereof).

With regard to the immunogenic polypeptide of SEQ ID NO:7, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 270 or fewer amino acids to SEQ ID NO:2, wherein the region includes SEQ ID NO:7. In preferred embodiments, the contiguous region will be “over 268,” “over 266,” “over 264,” “over 262,” “over 260,” “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:8, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 270 or fewer amino acids to SEQ ID NO:2, wherein the region includes SEQ ID NO:8. In preferred embodiments, the contiguous region will be “over 268,” “over 266,” “over 264,” “over 262,” “over 260,” “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:9, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 211 or fewer amino acids to SEQ ID NO:2, wherein the region includes SEQ ID NO:9. In preferred embodiments, the contiguous region will be “over 208,” “over 206,” “over 204,” “over 202,” “over 200,” “over 190,” “over 180,” “over 170,” “over 160,” “over 150,” “over 140,” “over 130,” “over 120,” “over 100,” “over 80,” “over 74,” “over 72,” “over 70,” “over 65,” “over 60,” “over 55,” “over 50,” “over 45,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 147,” “to 146,” “to 145,” “to 140,” “to 130,” “to 120,” “to 100,” “to 80,” “to 74,” “to 72,” “to 70,” “to 65,” “to 60,” “to 55,” “to 50,” “to 45,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:10, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 76 or fewer amino acids to SEQ ID NO:2, wherein the region includes SEQ ID NO:10. In preferred embodiments, the contiguous region will be “over 74,” “over 72,” “over 70,” “over 65,” “over 60,” “over 55,” “over 50,” “over 45,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 70,” “to 65,” “to 60,” “to 55,” “to 50,” “to 45,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:11, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 270 or fewer amino acids to SEQ ID NO:2, wherein the region includes SEQ ID NO:11. In preferred embodiments, the contiguous region will be “over 268,” “over 266,” “over 264,” “over 262,” “over 260,” “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:12, polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 270 or fewer amino acids to SEQ ID NO:2, wherein the region includes SEQ ID NO:12. In preferred embodiments, the contiguous region will be “over 268,” “over 266,” “over 264,” “over 262,” “over 260,” “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:9, additional polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) to SEQ ID NO:2, wherein the region includes SEQ ID NO:9 and extends no more than 47 amino acids upstream of SEQ ID NO:9. In preferred embodiments, the region of contiguous alignment will extend no more than 46, 44, 42, 40, 35, 30, 25, 20, 15, 10, 8, 7, 5, 4, 3, 2, or 1 amino acid(s) upstream. In some embodiments, the region of contiguous alignment will begin with the N-terminal end of SEQ ID NO:9.

With regard to the immunogenic polypeptide of SEQ ID NO:10, additional polypeptides of particular interest include polypeptides having a region of limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) to SEQ ID NO:2, wherein the region includes SEQ ID NO:10 and extends no more than 56 amino acids upstream. In preferred embodiments, the region of contiguous alignment will extend no more than 54, 52, 50, 48, 46, 44, 42, 40, 35, 30, 25, 20, 15, 10, 8, 7, 5, 4, 3, 2, or 1 amino acid(s) upstream. In some embodiments, the region of contiguous alignment will begin with the N-terminal end of SEQ ID NO:10.

In preferred embodiments, the polypeptides of the present invention will be capable of generating an immune response in a target organism such as a bird or a mammal, preferably a human subject. More preferably, the polypeptides will provide a target organism passive immunity and/or active immunity.

Additional embodiments of the polypeptides of the present invention may be found throughout the specification. By way of example, the polypeptides may further comprise targeting sequences such as secretion sequences, purification sequences, and fusion proteins including without limitation other immunogenic polypeptides, proteins that improve stability of the polypeptide, retention of the polypeptide within the subject, or antigenicity of the polypeptide.

In some embodiments, the polypeptide compositions of the present invention may additionally include other immunogenic polypeptides from GBS 80 (including without limitation polypeptides and polysaccharides) or other pathogens.

As described more fully below, additional aspects of the present invention include methods of using the foregoing polypeptides as (a) medicaments for treating or preventing infection due to Streptococcus bacteria; (b) diagnostics or immunodiagnostic assays for detecting the presence of Streptococcus bacteria or of antibodies raised against Streptococcus bacteria; and/or (c) reagents which can raise antibodies against Streptococcus bacteria.

Another aspect of the present invention includes methods of screening and/or testing peptides of the present invention for generation of an immune response, active immunization or passive immunization in a target organism. In some embodiments, the invention will involve contacting or administering the polypeptide composition of the present invention to the target organism and detecting antibodies in the target organism that recognize the polypeptide composition. In preferred embodiments, the target organism will be challenged with a Streptococcus bacterium to determine whether the target organism has active immunity or passive immunity. Such methods of screening may be applied to any of the compositions of the present invention including, without limitation, immunogenic polypeptides and pharmaceutical compositions for immunogenicity or antigenicity. A preferred embodiment of such screening methods includes providing an immunogenic polypeptide and screening the polypeptide for antigenicity or immunogenicity. Where more than one immunogenic polypeptide is to be screened, a criterion may be applied to select one or more immunogenic polypeptides for further use. Such criteria may be used to select among two or more immunogenic polypeptides, three or more immunogenic polypeptides, five or more immunogenic polypeptides, ten or more immunogenic polypeptides, or twenty or more immunogenic polypeptides.

Another aspect of the present invention is nucleic acids encoding any of the polypeptides of the present invention. In certain embodiments, such nucleic acids may be in an isolated or in recombinant form. In some embodiments, the nucleic acids encoding any of the foregoing polypeptides may be in a vector. In some embodiments, such nucleic acids may be operably linked to a promoter which preferably is operable in the host organism in which the polypeptide is to be expressed. In various embodiments, the promoter may be a constitutive promoter, a regulatable promoter, or an inducible promoter. Additional embodiments are described more fully below regarding expression vectors including nucleic acids of the present invention.

Another aspect of the present invention provides pharmaceutical compositions that include the polypeptides, antibodies, or nucleic acids of the present invention in a therapeutically effective amount (or an immunologically effective amount in a vaccine). In certain embodiments, the pharmaceutical compositions will be vaccines. The pharmaceutical vaccines may also have pharmaceutically acceptable carriers including adjuvants.

Additional aspects and embodiments may be found throughout the specification. The specification is not intended as a limitation of the scope of the present invention, but rather as examples of the aspects and embodiments of the present invention. One of skill in the art can infer additional embodiments from the description provided.

### BRIEF DESCRIPTION THE FIGURES

FIG. 1 shows the predicted fragments from the recombinantly produced GBS 80. The recombinantly produced protein has the N-terminal leader peptide removed (37 amino acids) and the C-terminal cell wall anchor and transmembrane region removed.

FIG. 2 shows the predicted mass-to-charge ratio for each of the predicted fragments identified in FIG. 1.

FIG. 3 shows a summary of western blot and FACs analysis conducted with six monoclonal antibodies directed to GBS 80 used to identify the immunogenic polypeptides herein.

FIG. 4 shows FACs analysis graphs of the six monoclonal antibodies and a polyclonal antibody serum.

FIG. 5 shows the general scheme used to identify fragments produced in partial digests of recombinantly produced GBS 80.

FIG. 6 shows western blots of partial Asp-N digests of recombinantly produced GBS. On the left is a western blot using the 9A4/77 monoclonal antibody and on the right is a western blot using the M3/88 monoclonal antibody.

FIG. 7 shows a Coomassie Blue stained SDS-PAGE of partial digests recombinantly produced GBS 80 using two different proteases, Asp-N and Arg-C. GBS 80 F and GBS 80 3 correspond to two different conformations of GBS 80 which have different protease sensitivities. The lanes are as labeled on the figure.

FIG. 8 shows a pair of western blots of the two conformers of GBS 80 partially digested with either Asp-N or Arg-C. On the left is a western blot using the 9A4/77 monoclonal antibody and on the right is a western blot using the M3/88 monoclonal antibody. The lanes are as labeled on the figure.

FIG. 9 shows an SDS-PAGE of the partial digests of boiled samples of GBS 80:1) an Arg-C partial digest of OBS 80 3, 2) an Arg-C partial digest of GBS 80 F, 3) an Asp-N partial digest of GBS 80 F, and 4) GBS 80 F (no digest). M indicates lanes with protein markers of the sizes indicated along the left of the gel image.

FIG. 10 shows the results of the western blot epitope mapping of the 9A4/77 monoclonal antibody and the M3/88 monoclonal antibody. The full length GBS 80 protein is shown schematically along the top with numbers indicating the amino acid position. Each protein fragment identified by MALDI-TOF from FIG. 9 is show below the full length GBS 80 protein with the corresponding fragment number. Along the left are two columns indicating which of the fragments were observed in the western blots with the two antibodies—N is 9A4/77 and C is M3/88. The two circles indicate the regions bound by each antibody.

FIG. 11 shows the sequence of the recombinantly produced GBS 80 protein (note: the recombinant GBS 80 has had the N-terminal leader sequence removed and replaced with a leading methionine residue, so amino acid 1 corresponds to 37 in the full length GBS 80 protein). Three immunogenic polypeptides are shown in the figure. The yellow region is recognized by 9A4/77. The cyan and green region is bound by M3/88 and the green region is the core region bound by M3/88.

FIG. 12 shows four western blots of the two conformers of GBS 80 partially digested with either Asp-N or Arg-C. The antibodies use to generate the western blots are indicated above the respective western blot. The lanes are as labeled on the figure.

FIG. 13 shows the sequence of the recombinantly produced GBS 80 protein (again note: the recombinant GBS 80 has had the N-terminal leader sequence removed and replaced with a leading methionine residue, so amino acid 1 corresponds to 37 in the full length GBS 80 protein). Three immunogenic polypeptides are shown in the figure. The yellow region is recognized by 19G4/78 and 19F6/77. The cyan and green region are bound by M1/77 and M2/77 while the green region represents the core region bound by the two antibodies.

FIG. 14 shows a schematic of the layout of a peptide microarray used to further identify immunogenic polypeptides in GBS 80. The control peptides are around the edges of the chip labeled with roman numerals I-VI. The GBS 80 peptides are numbered 1-80 as set out in Table 3 below (note: there are no peptides in positions 28-36).

FIG. 15 shows three peptide microarrays on a slide after fluorescent labeling. Control peptides are indicated with dashed circles and GBS 80 peptides are indicated with solid circles. Peptides number 73 and 75 were both bound by the monoclonal antibody 9A4/77 in all three arrays on the slide.

FIG. 16 shows the sequence of the recombinantly produced GBS 80 protein. Three immunogenic polypeptides are shown in the figure. The immunogenic polypeptide identified from the microarray epitope mapping is shown as cyan highlighting.

### DETAILED DESCRIPTION OF PREFERRED EMBODIMENTS

An aspect of the present invention provides fragments and sub fragments of the proteins and protein fragments disclosed in international patent applications WO04/041157 and WO05/028618 (the “International Applications”), wherein the fragments comprise at least one immunogenic polypeptide.

Thus, if the length of any particular protein or protein fragment sequence disclosed in the International Applications is x amino acids, the present invention provides fragments of at most x−1 amino acids of that protein. The fragment may be shorter than this (e.g., x−2, x−3, x−4, . . . ), and is preferably 100 amino acids or less (e.g., 90 amino acids, 80 amino acids etc.). The fragment may be as short as 3 amino acids, but is preferably longer (e.g., up to 5, 6, 7, 8, 9, 10, 12, 15, 20, 25, 30, 35, 40, 50, 75, or 100 amino acids).

Preferred fragments comprise the GBS 80 immunogenic polypeptides disclosed below, or sub-sequences thereof. The fragments may be longer than those disclosed below e.g., where a fragment runs from amino acid residue p to residue q of a protein, the invention also relates to fragments from residue (p−1), (p−2), or (p−3) to residue (q+1), (q+2), or (q+3), up to 1 amino acid less that the fragments disclosed in the International Applications.

The invention also provides polypeptides that are homologous (i.e., have sequence identity) to these fragments. Depending on the particular fragment, the degree of sequence identity is preferably greater than 50% (e.g., 60%, 70%, 80%, 85%, 90%, 95%, 96%, 97%, 98%, 99%, 99.5% or more). These homologous polypeptides include mutants and allelic variants of the fragments. Identity between the two sequences is preferably determined by the Smith-Waterman homology search algorithm as implemented in the MPSRCH program (Oxford Molecular), preferably using an affine gap search with parameters gap open penalty=12 and gap extension penalty=1.

The invention also provides proteins comprising one or more of the above-defined fragments.

The invention is subject to the proviso that it does not include within its scope proteins limited to any of the full length protein or protein fragment sequences disclosed in the International Applications (i.e., SEQ ID NOs: 1 and 2 of WO04/041157 and SEQ ID NOs: 1-9 of WO05/028618).

The proteins of the invention can, of course, be prepared by various means (e.g., recombinant expression, purification from cell culture, chemical synthesis etc.) and in various forms (e.g., native, C-terminal and/or N-terminal fusions etc.). They are preferably prepared in substantially pure form (i.e., substantially free from other GBS or host cell proteins, with the understanding that they may later be combined with antigens from GBS or other pathogens to create combination vaccines). Short polypeptides are preferably produced using chemical peptide synthesis.

According to a further aspect, the invention provides antibodies which recognize the fragments of the invention, with the proviso that the invention does not include within its scope antibodies which recognize any of the complete protein sequences in the International Applications. The antibodies may be polyclonal or monoclonal, and may be produced by any suitable means. Example 2 provides examples of monoclonal and polyclonal antibodies that recognize certain immunogenic polypeptides of the present invention.

The invention also provides proteins comprising peptide sequences recognized by these antibodies. These peptide sequences will, of course, include fragments of the GBS 80 protein and protein fragments in the International Applications, but will also include peptides that mimic the antigenic structure of the GBS 80 peptides when bound to immunoglobulin.

According to a further aspect, the invention provides nucleic acids encoding the fragments and proteins of the invention, with the proviso that the invention does not include within its scope nucleic acid encoding any of the full length protein or protein fragment sequences in the International Applications. The nucleic acids may be as short as 10 nucleotides, but are preferably longer (e.g., up to 10, 12, 15, 18, 20, 25, 30, 35, 40, 50, 75, or 100 nucleotides).

In addition, the invention provides nucleic acid comprising sequences homologous (i.e., having sequence identity) to these sequences. The degree of sequence identity is preferably greater than 50% (e.g., 60%, 70%, 80%, 85%, 90%, 95%, 96%, 97%, 98%, 99%, 99.5% or more). Furthermore, the invention provides nucleic acid which can hybridize to these sequences, preferably under “high stringency” conditions (e.g., at least one wash at 65° C. in a 0.1×SSC, 0.5% SDS for 15 minutes).

It should also be appreciated that the invention provides nucleic acid comprising sequences complementary to those described above (e.g., for antisense or probing purposes).

Nucleic acids according to the invention can, of course, be prepared in many ways (e.g., by chemical synthesis, from genomic or cDNA libraries, from the organism itself etc.) and can take various forms (e.g., single stranded, double stranded, vectors, probes etc.). In addition, the term “nucleic acid” includes DNA and RNA, and also their analogues, such as those containing modified backbones, and also peptide nucleic acids (PNA), etc. According to a further aspect, the invention provides vectors comprising nucleotide sequences of the invention (e.g., expression vectors) and host cells transformed with such vectors.

According to a further aspect, the invention provides compositions comprising protein, antibody, and/or nucleic acid according to the invention. These compositions may be suitable as vaccines, for instance, or as other prophylactic agents, or as diagnostic reagents, or as immunogenic compositions. Therefore, another aspect of the present invention includes the use of nucleic acid, protein, or antibody according to the invention in the manufacture of: (i) a medicament for treating or preventing infection due to Streptococcus bacteria; (ii) a diagnostic reagent for detecting the presence of Streptococcus bacteria or of antibodies raised against Streptococcus bacteria; and/or (iii) a reagent which can raise antibodies against Streptococcus bacteria. Said Streptococcus bacteria may be any species or strain (such as Streptococcus pyogenes and S. pneumonia) but are preferably the Lancefield-streptococci strains, more preferably the Lancefield group B strains and most preferably Streptococcus agalactiae, in each of the foregoing, the bacteria are limited to those having a GBS-type pilus and therefore a GBS 80 homolog. The invention also provides a method of treating a patient, comprising administering to the patient a therapeutically effective amount of nucleic acid, protein, and/or antibody according to the invention, According to further aspects, the invention provides various processes, for example:

A process for producing proteins of the invention is provided, comprising the step of culturing a host cell according to the invention under conditions which induce protein expression. A process for producing protein or nucleic acid of the invention is provided, wherein the protein or nucleic acid is synthesized in part or in whole using chemical means. A process for detecting polynucleotides of the invention is provided, comprising the steps of: (a) contacting a nucleic probe according to the invention with a biological sample under hybridizing conditions to form duplexes; and (b) detecting said duplexes. In preferred examples, the detection of the duplex involves amplification of the nucleic acid detected, more preferably through RT-PCR. A process for detecting proteins of the invention is provided, comprising the steps of: (a) contacting an antibody according to the invention with a biological sample under conditions suitable for the formation of an antibody-antigen complexes; and (b) detecting said complexes.

A summary of standard techniques and procedures which may be employed in order to perform the invention (e.g., to utilize the disclosed sequences for vaccination or diagnostic purposes) follows. This summary is not a limitation on the invention but, rather, gives examples which may be used, but which are not required.

### General

The practice of the present invention will employ, unless otherwise indicated, conventional techniques of molecular biology, microbiology, recombinant DNA, and immunology, which are within the skill of the art. Such techniques are explained fully in the literature. See, e.g., DNA Cloning, Volumes I and II (D. N Glover ed. 1985); Oligonucleotide Synthesis (MT Gait ed, 1984); Nucleic Acid Hybridization (B. D. Hames & S T Higgins eds. 1984); Transcription and Translation (B. D. Hames & S T Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), Handbook of Experimental Immunology, Volumes I-IV (D. M. Weir and C. C. Blackwell eds 1986), Remington's Pharmaceutical Sciences, Mack Publishing Company, Easton, Pa., 19th Edition (1995); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques An Intensive Laboratory Course, (Ream et al., eds., 1998, Academic Press); PCR (Introduction to Biotechniques Series), 2nd ed. (Newton & Graham eds., 1997, Springer Verlag); and Peters and Dalrymple, Fields Virology (2d ed), Fields et al. (eds.), B. N. Raven Press, New York, N.Y.

Standard abbreviations for nucleotides and amino acids are used in this specification.

All publications, patents, and patent applications cited herein are incorporated in full by reference.

### DEFINITIONS

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g., a composition “comprising” X may consist exclusively of X or may include something additional to X, such as X+Y.

The term “antigenic determinant” includes B-cell epitopes and T-cell epitopes.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a meningococcal sequence is heterologous to a mouse host cell. A further example would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

The term “a polypeptide having a region of limited, contiguous sequence identity of at least X percent over Y {or fewer} amino acids to SEQ ID NO:2, wherein the region includes SEQ ID NO:Z” as used herein means that the polypeptide has a percent identity of at least X percent (e.g., at least 50%, 60%, 70%, 80%, 85%, 90%, 95%, 96%, 97%, 98%, 99%, or 99.5%) when compared to SEQ ID NO:2, and the region of alignment in SEQ ID NO:2 includes SEQ ID NO:Z and is limited and contiguous. In the context of this phrase, contiguous means that when the polypeptide's sequence is aligned with SEQ ID NO:2 there are no gaps in the alignment or if there are, the amino acids across the gap are considered non-identical amino acids for the purpose of calculating the percent identity. In the context of this phrase, limited means that the polypeptide may be longer than Y amino acids, but that the polypeptide when aligned to the sequence of GBS 80 will have not have a region of alignment that is longer than Y amino acids. For the avoidance of doubt, where the region of alignment is flanked by amino acids that are not conserved, they are not included in the calculation of the length Y even if they could be included and still meet the percent identity. For example, the term “a polypeptide having a region of limited contiguous sequence identity of at least 90 percent over 100 amino acids to SEQ ID NO:2, wherein the region includes SEQ ID NO:7” would include a polypeptide that has a contiguous region of 100 amino acids that has 97% identity to GBS 80 (SEQ ID NO:2) and includes SEQ ID NO:7 even when the polypeptide is longer than 100 amino acids as long as the flanking amino acids are not conserved even though the flanking amino acids could be included and still be at least 90 percent identical (i.e., a pair of sequences that are 102 amino acids in length and have 97 conserved amino acids would have a 95% identity). Thus, the this term would include polypeptides that have additional sequences fused to the immunogenic polypeptide such as signal peptides, additional epitopes (including other epitopes from GBS 80 as long as they are not contiguous with the immunogenic polypeptide or are within the contiguous region), and other proteins and polypeptides that one of skill in the art may desire.

The term “a polypeptide having a region of limited, contiguous sequence identity of at least X percent to SEQ ID NO:2, wherein the region includes SEQ ID NO:Y and extends no more than Z amino acids {upstream/downstream} of SEQ ID NO:Y” as used herein means that the polypeptide has a region that is at least X percent identical (e.g., at least 50%, 60%, 70%, 80%, 85%, 90%, 95%, 96%, 97%, 98%, 99%, or 99.5%) when compared to SEQ ID NO:2, and the region of alignment in SEQ ID NO:2 includes SEQ ID NO:Y and is limited and contiguous. In the context of this phrase, contiguous means that when the polypeptide's sequence is aligned with SEQ ID NO:Y there are no gaps in the alignment or if there are, the amino acids across the gap are considered non-identical amino acids for the purpose of calculating the percent identity. In the context of this phrase, limited means that the polypeptide may extend upstream (i.e., N-terminal to SEQ ID NO:Y) or downstream (i.e., C-terminal to SEQ ID NO:Y) longer than Z amino acids, but that the polypeptide when aligned to the sequence of GBS 80 will have not have a region of alignment upstream or downstream, respectively, that extends more than Z amino acids from SEQ ID NO:Y. For the avoidance of doubt, where the region of alignment is flanked by amino acids that are not conserved, they are not included in the calculation of the length Z even if they could be included and still meet the percent identity. For example, the term “a polypeptide having a region of limited, contiguous sequence identity of at least 90 percent to SEQ ID NO:2 wherein the region includes SEQ ID NO:7 and extends no more than 50 amino acids upstream of SEQ IS NO:7” would include a polypeptide that has a contiguous region of 50 amino acids immediately upstream of the region that is identical to SEQ ID NO:7 that has 97% identity to GBS 80 and even when the polypeptide extends upstream further than 50 amino acids as long as the amino acids immediately upstream of the 50 amino acid stretch are not conserved even though the flanking amino acids could be included and still be at least 90 percent identical.

### GBS 80

GBS 80 refers to a putative cell wall surface anchor family protein. The nucleotide and amino acid sequences of GBS 80 sequenced from serotype V isolated strain 2603 V/R are set forth in Ref. 2 as SEQ ID 8779 and SEQ ID 8780. These sequences are also set forth below as SEQ ID NOS 1 and 2:

| SEQ ID NO. 1 | |
|---|---|
| ATGAAATTATCGAAGAAGTTATTGTTTTCGGCTGCTGTTTTAACAATGGTG | |
| GCGGGGTCAACTGTTGAACCAGTAGCTCAGTTTGCGACTGGAATGAGTATTGTAA | |
| GAGCTGCAGAAGTGTCACAAGAACGCCCAGCGAAAACAACAGTAAATATCTATA | |
| AATTACAAGCTGATAGTTATAAATCGGAAATTACTTCTAATGGTGGTATCGAGAA | |
| TAAAGACGGCGAAGTAATATCTAACTATGCTAAACTTGGTGACAATGTAAAAGG | |
| TTTGCAAGGTGTACAGTTTAAACGTTATAAAGTCAAGACGGATATTTCTGTTGAT | |
| GAATTGAAAAAATTGACAACAGTTGAAGCAGCAGATGCAAAAGTTGGAACGATT | |
| CTTGAAGAAGGTGTCAGTCTACCTCAAAAAACTAATGCTCAAGGTTTGGTCGTCG | |
| ATGCTCTGGATTCAAAAAGTAATGTGAGATACTTGTATGTAGAAGATTTAAAGAA | |
| TTCACCTTCAAACATTACCAAAGCTTATGCTGTACCGTTTGTGTTGGAATTACCAG | |
| TTGCTAACTCTACAGGTACAGGTTTCCTTTCTGAAATTAATATTTACCCTAAAAAC | |
| GTTGTAACTGATGAACCAAAAACAGATAAAGATGTTAAAAAATTAGGTCAGGAC | |
| GATGCAGGTTATACGATTGGTGAAGAATTCAAATGGTTCTTGAAATCTACAATCC | |
| CTGCCAATTTAGGTGACTATGAAAATTTGAAATTACTGATAAATTTGCAGATGGC | |
| TTGACTTATAAATCTGTTGGAAAATCAAGATTGGTTCGAAAACACTGAATAGAGA | |
| TGAGCACTACACTATTGATGAACCAACAGTTGATAACCAAAATACATTAAAAATT | |
| ACGTTTAAACCAGAGAAATTTAAAGAAATTGCTGAGCTACTTAAAGGAATGACC | |
| CTTGTTAAAAATCAAGATGCTCTTGATAAAGCTACTGCAAATACAGATGATGCGG | |
| CATTTTTGGAAATTCCAGTTGCATCAACTATTAATGAAAAAGCAGTTTTAGGAAA | |
| AGCAATTGAAAATACTTTTGAACTTCAATATGACCATACTCCTGATAAAGCTGAC | |
| AATCCAAAACCATCTAATCCTCCAAGAAAACCAGAAGTTCATACTGGTGGGAAA | |
| CGATTTGTAAAGAAAGACTCAACAGAAACACAAACACTAGGTGGTGCTGAGTTT | |
| GATTTGTTGGCTTCTGATGGGACAGCAGTAAAATGGACAGATGCTCTTATTAAAG | |
| CGAATACTAATAAAAACTATATTGCTGGAGAAGCTGTTACTGGGCAACCAATCA | |
| AATTGAAATCACATACAGACGGTACGTTTGAGATTAAAGGTTTGGCTTATGCAGT | |
| TGATGCGAATGCAGAGGGTACAGCAGTAACTTACAAATTAAAAGAAACAAAAGC | |
| ACCAGAAGGTTATGTAATCCCTGATAAAGAAATCGAGTTTACAGTATCACAAAC | |
| ATCTTATAATACAAAACCAACTGACATCACGGTTGATAGTGCTGATGCAACACCT | |
| GATACAATTAAAAACAACAAACGTCCTTCAATCCCTAATACTGGTGGTATTGGTA | |
| CGGCTATCTTTGTCGCTATCGGTGCTGCGGTGATGGCTTTTGCTGTTAAGGGGAT | |
| GAAGCGTCGTACAAAAGATAAC | |
| SEQ ID NO: 2 | |
| MKLSKKLLFS AAVLTMVAGS TVEPVAQFAT GMSIVRAAEV SQERPAKTTV | |
| NIYKLQADSY KSEITSNGGI ENKDGEVISN YAKLGDNVKG LQGVQFKRYK | 100 |
| VKTDISVDEL KKLTTVEAAD AKVGTILEEG VSLPQKTNAQ GLVVDALDSK | |
| SNVRYLYVED LKNSPSNITK AYAVPFVLEL PVANSTGTGF LSEINIYPKN | 200 |
| VVTDEPKTDK DVKKLGQDDA GYTIGEEFKW FLKSTIPANL GDYEKFEITD | |
| KFADGLTYKS VGKIKIGSKT LNRDEHYTID EPTVDNQNTL KITFKPEKFK | 300 |
| EIAELLKGMT LVKNQDALDK ATANTDDAAF LEIPVASTIN EKAVLGKAIE | |
| NTFELQYDHT PDKADNPKPS NPPRKPEVHT GGKRFVKKDS TETQTLGGAE | 400 |
| FDLLASDGTA VKWTDALIKA NTNKNYIAGE AVTGQPIKLK SHTDGTFEIK | |
| GLAYAVDANA EGTAVTYKLK ETKAPEGYVI PDKEIEFTVS QTSYNTKPTD | 500 |
| ITVDSADATP DTIKNNKRPS IPNTGGIGTA IFVAIGAAVM AFAVKGMKRR | |
| TKDN | |

GBS 80 contains an N-terminal leader or signal sequence region which is indicated by the underlined sequence at the beginning of SEQ ID NO: 2 above. GBS 80 also contains a C-terminal transmembrane region which is indicated by the underlined sequence near the end of SEQ ID NO: 2 above. In preferred embodiments, the immunogenic polypeptides will have one or more amino acids from the transmembrane region and/or a cytoplasmic region removed to improve solubility of the antigen. GBS 80 contains an amino acid motif indicative of a cell wall anchor: SEQ ID NO: 31PNTG (shown in italics in SEQ ID NO: 2 above). In some recombinant host cell systems, it may be preferable to remove this motif to facilitate secretion of a recombinant GBS 80 protein from the host cell. Accordingly, in preferred embodiments of the immunogenic polypeptides of GBS 80 for use in the invention, the transmembrane and/or cytoplasmic regions and the cell wall anchor motif are not included in the immunogenic polypeptides. Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

A recombinantly produced GBS 80 fragment was used in the examples set out below that has the N-terminal leader sequence removed and replaced with an N-terminal methionine and the C-terminal cell wall anchor and transmembrane regions removed. The sequence of the recombinantly produced GBS 80 fragment is set out below:

| (SEQ ID NO: 4) | |
|---|---|
| MAEVSQERPA KTTVNIYKLQ ADSYKSEITS NGGIENKDGE VISNYAKLGD | |
| NVKGLQGVQF KRYKVKTDIS VDELKKLTTV EAADAKVGTI LEEGVSLPQK | 100 |
| TNAQGLVVDA LDSKSNVRYL YVEDLKNSPS NITKAYAVPF VLELPVANST | |
| GTGFLSEINI YPKNVVTDEP KTDKDVKKLG QDDAGYTIGE EFKWFLKSTI | 200 |
| PANLGDYEKF EITDKFADGL TYKSVGKIKI GSKTLNRDEH YTIDEPTVDN | |
| QNTLKITFKP EKFKEIAELL KGMTLVKNQD ALDKATANTD DAAFLEIPVA | 300 |
| STINEKAVLG KAIENTFELQ YDHTPDKADN PKPSNPPRKP EVHTGGKRFV | |
| KKDSTETQTL GGAEFDLLAS DGTAVKWTDA LIKANTNKNY IAGEAVTGQP | 400 |
| IKLKSHTDGT FEIKGLAYAV DANAEGTAVT YKLKETKAPE GYVIPDKEIE | |
| FTVSQTSYNT KPTDITVDSA DATPDTIKNN KRPS | |

As described above, the invention includes fragments of a GBS 80 immunogenic polypeptide. The GBS 80 immunogenic polypeptides include the immunogenic epitopes of the cited GBS antigens may be used in the compositions of the invention.

Applicants have identified a particularly immunogenic fragment of the GBS 80 protein. This immunogenic fragment is located towards the N-terminus of the protein and is underlined in the GBS SEQ ID NO: 2 sequence below. The underlined fragment is set forth below as SEQ ID NO: 5.

SEQ ID NO: 2
MKLSKKLLFSAAVLTMVAGSTVEPVAQFATGMSIVRAAEVSQERPAKTT
VNIYKLQADSYKSEITSNGGIENKDGEVISNYAKLGDNVKGLQGVQFKR
YKVKTDISVDELKKLTTVEAADAKVGTILEEGVSLPQKTNAQGLVVDAL
DSKSNVRYLYVEDEKNSPSNITYAVPFVLELPVANSTGTGFSEINIYPK
NWTDEPKTDKDVKKLGQDDAGYTIGEEFKFKSTIPANLGDYEKFEITDK
FADGLTYKSVGKIKIGSKTLNRDEHYTIDEPTVDNQNTLKITFKPEKFK
EIAELLKGMTEVKNQDALDKATANTDDAAFLEIPVASTINEKAVLGKAI
ENTFELQYDHTPDKADNPKPSNPPRKPEVHTGGKRFVKKDSTETQTLGG
AEFDLLASDGTAVKTDALIKANTNKNYIAGEAVTGQPIKKSHTDGTFEI
KGLAYAVDANAEGTAVTYKKETKAPEGYVIPDKEIEFTVSQTSYNTKPT
DITVDSADATPDTIKNNKRPSIPNTGGIGTAIFVAIGAAVMAFAVKGMK
RRTKDN
SEQ ID NO: 5
AEVSQERPAKTTVNIYKLQADSYKSEITSNGGIENKDGEVISNYAKLGD
NVKGLQGVQFKRYKVKTDISVDELKKLTTVEAADAKVGTILEEGVSLPQ
KTNAQGLVVDALDSKSNVRYLYVEDLKNSPSNITKAYAVPFVLELPVAN
STGTGFLSEINIYPKNWTDEPKTDKDVKKLGQDDAGYTIGEEFKWFLKS
TIPANLGDYEKFEITDKFADGLTYKSVGKIKIGSKTLNRDEHYTIDEPT
VDNQNTLKITFKPEKFKEIAELLKG

Two of the immunogenic polypeptides identified in Example 2 are shown in SEQ ID NO:5 above. SEQ ID NO: 7 is underlined and SEQ ID NO: 8 is highlighted in bold.

SEQ ID NO: 7
DGEVISNYAKLGDNVKGLQGVQFKRYKVKTDISVDELKKLTTVEAADAK
VGTILEEGVSLPQKTNAQGLVVDALDSKSNVR
SEQ ID NO: 8
GLQGVQFKRYKVKTDISVDELKKLTTVEAADAKVGTILEEGVSLPQKTN
AQGLVVDALDSKSNVR

The immunogenicity of the protein encoded by SEQ ID NO: 5 was compared against PBS, GBS whole cell, GBS 80 (full length) and another fragment of GBS 80, located closer to the C terminus of the peptide (SEQ ID NO: 6, below).

SEQ ID NO: 6
MTLVKNQDALDKATANTDDAAFLEIPVASTINEKAVLGKAIENTFELQY
DGTPDKADNPKPSNPPRKPEVHTGGKRFVKKDSTETQTLGGAEFDLLAS
DGTAVKWTDALIKANTNKNYIAGEAVTGQPIKLKSHTDGTFEIKGLAYA
VDANAEGTAVTYKLKETIAPEGYVIPDKEIEFTVSQTSYNTKPTDITVD
SADATPDTIKNNKRPS

Two of the immunogenic polypeptides identified in Example 2 are shown in SEQ ID NO: 6 above. SEQ ID NO: 9 is underlined and SEQ ID NO: 10 is highlighted in bold.

SEQ ID NO: 9
YDGTPDKADNPKPSNPPRKPEVHTGGKRFV
SEQ ID NO: 10
NPKPSNPPR

The peptide array epitope mapping described in Example 3 identified two additional immunogenic polypeptides—DALDSKSNVRYLY (SEQ ID NO:11) and SNVRYLYVEDLKN (SEQ ID NO:12).

### GBS 80 Immunogenic Polypeptides

As discussed above, one embodiment of the invention provides an immunogenic composition comprising an immunogenic polypeptide from GBS 80 or a fragment thereof, wherein said immunogenic polypeptide is a fragment of GBS 80 that includes one of the regions identified in this application and may include additional portions of OBS 80. Of particular interest are the immunogenic polypeptides of SEQ ID NOs: 7-12. The length of the fragment may vary depending on the amino acid sequence of the specific immunogenic polypeptide, but the fragment is preferably at least 7 consecutive amino acids, (e.g. 8, 10, 12, 14, 16, 18, 20, 25, 30, 35, 40, 50, 60, 70, 80, 90, 100, 150, 200 or more).

The immunogenic polypeptides may include polypeptide sequences having sequence identity to the identified immunogenic polypeptides. The degree of sequence identity may vary depending on the amino acid sequence in question, but is preferably greater than 50% (e.g. 60%, 65%, 70%, 75%, 80%, 85%, 90%, 91%, 92%, 93%, 94%, 95%, 96%, 97%, 98%, 99%, 99.5% or more). Polypeptides having sequence identity include homologs, orthologs, allelic variants and functional mutants of the identified GBS 80 immunogenic polypeptides. Typically, 50% identity or more between two proteins is considered to be an indication of functional equivalence. Identity between proteins is preferably determined by the Smith-Waterman homology search algorithm as implemented in the MPSRCH program (Oxford Molecular), using an affinity gap search with parameters gap open penalty=12 and gap extension penalty=1. The immunogenic polypeptides may include polypeptide sequences encoded by nucleic acid sequences that hybridize under high stringency wash conditions (see below for representative conditions) to nucleic acids encoding an identified immunogenic polypeptide (especially SEQ ID NO: 7-12 or an antigenic fragment thereof).

With regard to the immunogenic polypeptide of SEQ ID NO:7, polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 259 or fewer amino acids to SEQ ID NO:2 including SEQ ID NO:7. In preferred embodiments, the contiguous region will be “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:8, polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 259 or fewer amino acids to SEQ ID NO:2 including SEQ ID NO:8. In preferred embodiments, the contiguous region will be “over 250,” “over 240,” “over 230,” “over 220,” “over 210,” “over 200,” “over 180,” “over 160,” “over 140,” “over 120,” “over 100,” “over 80,” “over 60,” “over 50,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 200,” “to 180,” “to 160,” “to 140,” “to 120,” “to 100,” “to 80,” “to 60,” “to 50,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:9, polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 148 or fewer amino acids to SEQ ID NO:2 including SEQ ID NO:9. In preferred embodiments, the contiguous region will be “over 147,” “over 146,” “over 145,” “over 140,” “over 130,” “over 120,” “over 100,” “over 80,” “over 74,” “over 72,” “over 70,” “over 65,” “over 60,” “over 55,” “over 50,” “over 45,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 147,” “to 146,” “to 145,” “to 140,” “to 130,” “to 120,” “to 100,” “to 80,” “to 74,” “to 72,” “to 70,” “to 65,” “to 60,” “to 55,” “to 50,” “to 45,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:10, polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) over 76 or fewer amino acids to SEQ ID NO:2 including SEQ ID NO:10. In preferred embodiments, the contiguous region will be “over 74,” “over 72,” “over 70,” “over 65,” “over 60,” “over 55,” “over 50,” “over 45,” “over 40,” “over 35,” “over 30,” “over 27,” “over 23,” “over 20,” “over 18,” “over 16,” “over 14,” “over 13,” “over 11,” “over 10,” “over 9,” “over 8,” or “over 7.” In certain embodiments, a lower limit on the region of contiguous identity is desired and the phrase “or fewer” may be replaced with “to 70,” “to 65,” “to 60,” “to 55,” “to 50,” “to 45,” “to 40,” “to 35,” “to 30,” “to 27,” “to 23,” “to 20,” “to 18,” “to 16,” “to 14,” “to 13,” “to 11,” “to 10,” “to 9,” “to 8,” or “to 7.” One of skill in the art will appreciate that any pair-wise combination of limits may be selected as desired and that the same upper and lower limit may be selected to specify the desired length of the region of contiguous alignment.

With regard to the immunogenic polypeptide of SEQ ID NO:9, additional polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) percent to SEQ ID NO:2 including SEQ ID NO:9 extending no more than 47 amino acids upstream. In preferred embodiments, the region of contiguous alignment will extend no more than 46, 44, 42, 40, 35, 30, 25, 20, 15, 10, 8, 7, 5, 4, 3, 2, or 1 amino acid(s) upstream. In some embodiments, the region of contiguous alignment will begin with the N-terminal end of SEQ ID NO:9.

With regard to the immunogenic polypeptide of SEQ ID NO:10, additional polypeptides of particular interest include polypeptides having limited, contiguous sequence identity of at least 50 percent (or with increasing preference at least 60%, at least 70%, at least 80%, at least 85%, at least 90%, at least 95%, at least 96%, at least 97%, at least 98%, at least 99%, or at least 99.5%) percent to SEQ ID NO:2 including SEQ ID NO:10 extending no more than 56 amino acids upstream. In preferred embodiments, the region of contiguous alignment will extend no more than 54, 52, 50, 48, 46, 44, 42, 40, 35, 30, 25, 20, 15, 10, 8, 7, 5, 4, 3, 2, or 1 amino acid(s) upstream. In some embodiments, the region of contiguous alignment will begin with the N-terminal end of SEQ ID NO:10.

### Expression Systems

The GBS 80 immunogenic polypeptide nucleotide sequences can be expressed in a variety of different expression systems; for example those used with mammalian cells, baculoviri, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g., structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation (Sambrook et. (1989) Expression of Cloned Genes in Mammalian Cells. In Molecular Cloning: A Laboratory Manual, 2nd ed.).

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothionein gene, also provide useful promoter sequences, Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter (Maniatis et al. (1987) Science 236:1237; Alberts et al. (1989) Molecular Biology of the Cell, 2nd ed.). Enhancer elements derived from viruses may be particularly useful, because they usually have a broader host range. Examples include the SV40 early gene enhancer (Dijkema et al (1985) EMBO J. 4:7611) and the enhancer/promoters derived from the long terminal repeat (LTR) of the Rous Sarcoma Virus (Gorman et al. (1982b) Proc. Natl. Acad. Sci. 79:6777) and from human cytomegalovirus (Boshart et al. (1985) Cell 41:5211). Additionally, some enhancers are regulatable and become active only in the presence of an inducer, such as a hormone or metal ion (Sassone-Corsi and Borelli (1986) Trends Genet. 2:215; Maniatis et al. (1987) Science 236:1237).

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells, Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus tripartite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al. (1985) Cell 41:349; Proudfoot and Whitelaw (1988) Termination and 3′ end processing of eukaryotic RNA. In Transcription and splicing (ed. B. D. Hames and D. M, Glover); Proudfoot (1989) Trends Biochem. Sci. 14:1051). These sequences direct the transcription of an mRNA which can be translated into the polypeptide encoded by the DNA. Examples of transcription terminater/polyadenylation signals include those derived from SV40 (Sambrook et al (1989) Expression of cloned genes in cultured mammalian cells. In Molecular Cloning: A Laboratory Manual).

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g., plasmids) capable of stable maintenance in a host, such as mammalian cells or bacteria. Mammalian replication systems include those derived from animal viruses, which require trans-acting factors to replicate. For example, plasmids containing the replication systems of papovaviri, such as SV40 (Gluzman (1981) Cell 23:1751) or polyomavirus, replicate to extremely high copy number in the presence of the appropriate viral T antigen. Additional examples of mammalian replicons include those derived from bovine papillomavirus and Epstein-Barf virus. Additionally, the replicon may have two replication systems, thus allowing it to be maintained, for example, in mammalian cells for expression and in a prokaryotic host for cloning and amplification. Examples of such mammalian-bacteria shuttle vectors include pMT2 (Kaufman et al. (1989) Mol. Cell. Biol. 9:946) and pHEBO (Shimizu et al. (1986) Mol. Cell. Biol. 6:10741). The transformation procedure used depends upon the host to be transformed. Methods for introduction of heterologous polynucleotides into mammalian cells are known in the art and include dextran-mediated transfection, calcium phosphate precipitation, protoplast fusion, electroporation, encapsulation of the polynucleotide(s) in 11posomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g., Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media, After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and Methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g., plasmids) capable of stable maintenance in a host, such as a bacterium, The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT (Luckow and Summers, Virology (1989) 17:31).

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein (Friesen et al., (1986) The Regulation of Baculovirus Gene Expression, in: The Molecular Biology of BaculovirUses (ed. Walter Doerfler); EPO Publ. Nos. 127 839 and 155 476) and the gene encoding the p10 protein (Vlak et al, (1988), J. Gen. Virol. 69:765).

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell post-translational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon (Maeda et al., (1985), Nature 315:592); human gastrin-releasing peptide (Lebacq-Verheyden et al., (1988), Molec. Cell. Biol. 8:3129); human IL-2 (Smith et al., (1985) Proc. Nat'l Acad. Sci. USA, 82:8404); mouse IL-3 (Miyajima et al., (1987) Gene 58:273); and human glucocerebrosidase (Martin et al. (1988) DNA, 7:99), can also be used to provide for secretion in insects.

A recombinant polypeptide or polyprotein may be expressed intracellularly or, if it is expressed with the proper regulatory sequences, it can be secreted. Good intracellular expression of non-fused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art, (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene (Miller et al., (1989), Bioessays 4:91). The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wildtype virus, the transfection supernatant is plagued onto a monolayer of insect cells by techniques known to those skilled in the ‘art. Namely, the plaques are screened under the light microscope for the presence (indicative of wild-type virus) or absence (indicative of recombinant virus) of occlusion bodies (Current Protocols in Microbiology, Vol. 2 (Ausubel et al. eds) at 16.8 (Supp. 10, 1990); Summers and Smith, supra; Miller et al. (1989)).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g., Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g., HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g., proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture have been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); and Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038 (1990); Maas et al., EMBO J. 9:3447-3452 (1990); Benkel and Hickey, Proc. Natl. Acad. Sci. 84:1337-1339 (1987)

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11 (2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eukaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code (Reed and Maniatis, Cell 41:95-105, 1985).

The vector can be microinjected directly into plant cells by use of micropipettes to mechanically transfer the recombinant DNA (Crossway, Mol. Gen. Genet, 202:179-185, 1985). The genetic material may also be transferred into the plant cell by using polyethylene glycol (Krens, et al., Nature, 296, 72-74, 1982). Another method of introduction of nucleic acid segments is high velocity ballistic penetration by small particles with the nucleic acid either within the matrix of small beads or particles, or on the surface (Klein, et al., Nature, 327, 70-73, 1987 and Knudsen and Muller, 1991, Planta, 185:330-336) teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation (Fromm et al., Proc. Natl. Acad. Sci. USA 82:5824, 1985). In this technique, plant protoplasts are electroporated in the presence of plasmids containing the gene construct. Electrical impulses of high field strength reversibly permeablize membranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trzfolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (e.g., a structural gene) into mRNA, A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (Raibaud et al. (1984) Annu. Rev. Genet. 18:173). Regulated expression may therefore either be positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) (Chang et al. (1977) Nature 198:1056), and maltose. Additional examples include promoter sequences derived from biosynthetic enzymes such as tryptophan (trp) (Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775); and the β-lactamase (bla) promoter system (Weissmann (1981) “The cloning of interferon and other mistakes.” In Interferon 3 (ed. 1. Gresser)). The bacteriophage lambda PL (Shimatake et al. (1981) Nature 292:128) and T5 (U.S. Pat. No. 4,689,406) promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter (U.S. Pat. No. 4,551,4331). For example, the tac promoter is a hybrid trp-lac promoter comprised of both trp promoter and lac operon sequences that is regulated by the lac repressor (Amann et al. (1983) Gene 25:167; de Boer et al. (1983) Proc. Natl. Acad. Sci. 80:21). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al. (1986) J. Mol. Biol. 189:113; Tabor et al. (1985) Proc Natl. Acad. Sci. 82:1074). In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon (Shine et al. (1975) Nature 254:34). The SD sequence is thought to promote binding of mRNA to the ribosome by the pairing of bases between the SD sequence and the 3′ end of E. coli 16S rRNA (Steitz et al. (1979) “Genetic signals and nucleotide sequences in messenger RNA.” In Biological Regulation and Development: Gene Expression (ed. R. F. Goldberger)). To express eukaryotic genes and prokaryotic genes with weak ribosome-binding site (Sambrook et al. (1989) “Expression of cloned genes in Escherichia coli.” In Molecular Cloning: A Laboratory Manual).

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene (Nagai et al. (1984) Nature 309:8101). Fusion proteins can also be made with sequences from the lacZ (Jia et al. (1987) Gene 60:197), trpE (Allen et al. (1987) J. Biotechnol. 5:93; Makoff et al. (1989) J. Gen. Microbiol. 135:11), and Chey (EP-A-0 324 647) genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g., ubiquitin specific processing-protease) to cleave the ubiquitin from the foreign protein. Through this method, native foreign protein can be isolated (Miller et al. (1989) Bio/Technology 7:698).

Alternatively, foreign proteins can also be secreted from the cell by creating chimeric DNA molecules that encode a fusion protein comprised of a signal peptide sequence fragment that provides for secretion of the foreign protein in bacteria (U.S. Pat. No. 4,336,336). The signal sequence fragment usually encodes a signal peptide comprised of hydrophobic amino acids which direct the secretion of the protein from the cell. The protein is either secreted into the growth media (gram-positive bacteria) of into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) (Masui et al. (1983), in: Experimental Manipulation of Gene Expression; Ghrayeb et al. (1984) EMBO J. 3:2437) and the E. coli alkaline phosphatase signal sequence (phoA) (Oka et al. (1985) Proc. Natl. Acad. Sci. 82:7212). As an additional example, the signal sequence of the alpha-amylase gene from various Bacillus strains can be used to secrete heterologous proteins from B. subtilis (Palva et al. (1982) Proc. Natl. Acad. Sci. USA 79:5582; EP-A-0 244 042).

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E, coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g., plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome, For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline (Davies et al. (1978) Annu. Rev. Microbiol. 32:469). Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis (Palva et al. (1982) Proc. Natl. Acad. Sci. USA 79:5582; EP-A-0 036 259 and EP-A-0 063 953; WO 84/04541), Escherichia coli (Shimatake et al. (1981) Nature 292:128; Amann et al. (1985) Gene 40:183; Studier et al. (1986) J. Mol. Biol. 189:113; EP-AM 036 776, EPA-0 136 829 and EP-A-0 136 907), Streptococcus cremoris (Powell et al. (1988) Appl. Environ. Microbiol. 54:655), Streptococcus lividans (Powell et al. (1988) Appl. Environ. Microbiol. 54:655), and Streptomyces lividans (U.S. Pat. No. 4,745,056).

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See e.g., (Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus) (Miller et al. (1988) Proc. Natl. Acad. Sci. 85:856; Wang et al. (1990) J. Bacteriol. 172:949, Campylobacter), (Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S, Nicosia); Mandel et al. (1970) J. Mol. Biol. 53:159; Taketo (1988) Biochim. Biophys. Acta 949:318; Escherichia), (Chassy et al. (1987) FEMS Microbiol. Lett. 44:173, Lactobacillus), (Fiedler et al. (1988) Anal. Biochem 170:38, Pseudomonas), (Augustin et al. (1990) FEMS Microbiol. Lett. 66:203, Staphylococcus), (Barany et al. (1980) J. Bacteriol. 144:698; Harlander (1987) “Transformation of Streptococcus lactis by electroporation, in: Streptococcal Genetics (ed. J. Ferretti and R. Curtiss 111); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus).

v. Yeast Expression

Yeast expression systems are also known to one of ordinary skill in the art. A yeast promoter is any DNA sequence capable of binding yeast RNA polymerase and initiating the downstream 3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS, but may be enhanced with one or more UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences (Myanohara et al. (1983) Proc. Natl. Acad. Sci. USA 80:1).

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the AD112, GAL4, GALIO, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, (Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109).

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g., EP-A-0 196 056. Another example is a ubiquitin fusion protein. Such a fusion protein is made with the ubiquitin region that preferably retains a site for a processing enzyme (e.g., ubiquitin specific processing protease) to cleave the ubiquitin from the foreign protein. Through this method, therefore, native foreign protein can be isolated (e.g., WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EP-A-0 012 873; JPO. 62,096,086) and the A-factor gene (U.S. Pat. No. 4,588,684). Alternatively, leaders of non-yeast origin, such as an interferon leader, exist that also provide for secretion in yeast (EP-A-0 060 057).

A preferred class of secretion leader sequences is that which employs a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alpha factor. (e.g., see W 0 89/02463.) Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a prokaryotic host for cloning and amplification, Examples of such yeast-bacteria shuttle vectors include YEp24 (Botstein et al. (1979) Gene 8:17-24), pC1/1 (Brake et al. (1984) PNAS USA 81:4642-4646), and YRp17 (Stinchcomb et al. (1982) J. Mol. Biol. 158:157). In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Either a high or low copy number vector may be selected, depending upon the effect of the vector and the foreign protein on the host. See e.g., Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al. (1983) Methods in Enzymol. 101:228-245). An integrating vector may be directed to a specific locus in yeast by selecting the appropriate homologous sequence for inclusion in the vector. See On-Weaver et al., supra. One or more expression construct may integrate, possibly affecting levels of recombinant protein produced (Rine et al. (1983) Proc. Natl. Acad. Sci. USA 80:6750). The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRPI, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1; allows yeast to grow in the presence of copper ions (Butt et al. (1987) Microbiol, Rev. 51:351), Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans (Kurtz, et al. (1986) Mol. Cell. Biol. 6:142), Candida maltosa (Kunze, et al. (1985) J. Basic Microbiol. 25:141), Hansenula polymorpha (Gleeson, et al. (1986) J. Gen. Microbiol. 132:3459; Roggenkamp et al. (1986) Mol. Gen. Genet. 202:302), Kluyveromyces fragilis (Das, et al. (1984) J. Bacteriol. 158:1165), Kluyveromyces lactis (De Louvencourt et al. (1983) J. Bacteriol. 154:737; Van den Berg et al. (1990) BiolTechnology 8:135), Pichia guillerimondii (Kunze et al. (1985) J. Basic Microbiol. 25:141), Pichia pastoris (Cregg, et al. (1985) Mol. Cell. Biol. 5:3376; U.S. Pat. Nos. 4,837,148 and 4,929,555), Saccharomyces cerevisiae (Hinnen et al. (1978) Proc. Natl. Acad. Sci. USA 75:1929; Ito et al. (1983) J. Bacteriol. 153:163), Schizosaccharomyces pombe (Beach and Nurse (1981) Nature 300:706), and Yarrowia lipolytica (Davidow, et al. (1985) Curr. Genet. 10:39; Gaillardin, et al. (1985) Curr. Genet. 10:49).

Methods of introducing exogenous DNA into yeast hosts are well-known in the art, and usually include either the transformation of spheroplasts or of intact yeast cells treated with alkali cations. Transformation procedures usually vary with the yeast species to be transformed. See e.g., (Kurtz et al. (1986) Mol. Cell. Biol. 6:142; Kunze et al. (1985) J. Basic Microbiol. 25:141; Candida); (Gleeson et al. (1986) J. Gen. Microbiol. 132:3459; Roggenkamp et al. (1986) Mol. Gen. Genet. 202:302; Hansenula); (Das et al. (1984) J. Bacteriol. 158:1165; De Louvencourt et al. (1983) J. Bacteriol. 154:1165; Van den Berg et al. (1990) BiolTechnology 8:135; Kluyveromyces); (Cregg et al. (1985) Mol. Cell. Biol. 5:3376; Kunze et al, (1985) J. Basic Microbiol. 25:141; U.S. Pat. Nos. 4,837,148 and 4,929,555; Pichia); (Hinnen et al. (1978) Proc. Natl. Acad. Sci. USA 75; 1929; Ito et al. (1983) J. Bacteriol. 153:163; Saccharomyces); (Beach and Nurse (1981) Nature 300:706; Schizosaccharomyces); and (Davidow et al. (1985) Curr. Genet. 10:39; Gaillardin et al. (1985) Curr. Genet. 10:49; Yarrowia).

### Screens

Another aspect of the present invention includes screening of the immunogenic compositions. Such screening may be performed for a wide range of purposes including by way of example selecting more antigenic immunogenic polypeptides to maximize the immune response in the vaccine recipient, screening multi-component vaccine candidates for immune response to all of the components, screening immunogenic polypeptides for no or only limited side effects, and screening for any other characteristic one of skill in the art may desire, non-limiting examples of which may be found throughout the specification.

The immunogenicity of immunogenic polypeptides may be assayed by any method known to one skilled in the art. Typically, the presence (or absence), titres, affinities, avitidies, etc. of antibodies generated in vivo are tested by standard methods, such as, but not limited to, ELISA assays, by which the immunogenicity or antigenicity are tested on immunoglobulin present in the serum of an organism (or patient). Additional methods, such as generating T-cell hybridomas and measuring activation in the presence of antigen presenting cells (“APCs”) and antigen (Surman S et al., 2001 Proc. Natl. Acad. Sci. USA 98: 4587-92, below), examining labeled or unlabeled MHC presented peptides by chromatography, electorphoresis, and/or mass spectroscopy, T-cell activation assays, such as, but not limited to, T cell proliferation assays (Adorini L et al., 1988. J. Exp. Med. 168: 2091; So T. et al., 1996. Immunol. Let. 49: 91-97) and IL-2 production by proliferative response assays of CTLL-2 cells (Gillis S et al., 1978. J. Immunol. 120: 2027; So T. et al., 1996. Immunol. Let. 49: 91-97), and many others may be applied to determine more specific aspects of an immune response, or the lack thereof, such as, for example, the identity of the immunogenic T cell epitope of the antigen.

As non-limiting, specific examples, in vitro T cell assays may be carried out whereby the polypeptide, protein, or protein complex can be processed and presented in the groove of MHC molecules by appropriate antigen-presenting cells (APCs) to syngeneic T cells. T cell responses may be measured by simple proliferation measurements or by measuring release of specific cytokine by activated cells; APCs may be irradiated or otherwise treated to prevent proliferation to facilitate interpretation of the results of such assays. In order to determine the immunogenicity of an epitope in the context of different MHC allotypes, in vivo assays using syngeneic APCs and T-cells of a range of allotypes may be carried out to test for T cell epitopes in a range of individuals or patients.

Alternatively, transgenic animals expressing MHC molecules from human (or any other species of interest) maybe used to assay for T cell epitopes; in a preferred embodiment this assay is carried out in transgenic animals in which the endogenous MHC repertoire has been knocked out and, better yet, in which one or more other accessory molecules of the endogenous MHC/T cell receptor complex have also been replaced with human molecules (or molecules of any other species of interest), such as, for example, the CD4 molecule.

Furthermore, to detect anti-protein/antigen/immunogenic polypeptide antibodies directly in vivo, for example in clinical and animal studies, ELISA assays, such as, for example solid phase indirect ELISA assays, may be used to detect binding of antibodies. In one specific embodiment, microtiter plates are incubated with the immunogenic polypeptide of interest at an appropriate concentration and in a suitable buffer. After washes with an appropriate washing solution, such as, for example PBS (pH 7.4), PBS containing 1% BSA and 0.05% Tween 20, or any other such solution as may be appropriate, serum samples are diluted, for example in PBS/BSA, and equal volumes of the samples are added in duplicate to the wells. The plates are incubated, and after additional washes, for example with PBS, anti-immunoglobulin antibodies coupled/conjugated to a reporter, such as a radioactive isotope or alkaline phosphatase, are added to each well at an appropriate concentration, and incubated. The wells are then washed again, and for example, in the case of use of alkaline phosphatase as a reporter, the enzyme reaction is carried our using a colorometric substrate, such as p-nitrophenyl phosphate in diethanolamine buffer (pH 9.8), absorbance of which can be read at 405 nm, for example, in an automatic ELISA reader (e.g. Multiskan PLUS; Labsystems).

As an additional non-limiting example, to detect antibodies in the serum of patients and animals, immunoblotting can also be applied. In one specific embodiment, an appropriate amount of the immunogenic polypeptide of interest per samples/lane is run on gels (e.g. polyacrylamide), under reducing and/or nonreducing conditions, and the polypeptide is transferred to a membrane, such as, for example, PVDF membranes; any other method to separate proteins by size can be used followed by transfer of the polypeptide to a membrane. The membranes are blocked, for example, using a solution of 5% (w/v) milk powder in PBS. In another embodiment, purified immunogenic polypeptide may be applied to the membrane. The blots are then incubated with serum samples at varying dilutions in the blocking solution (before and after injection regimen) and control anti-antigen, so far as such samples are available. The blots will be washed four times with an appropriate washing solution, and further incubated with reporter-conjugated anti-immunoglobulin at a appropriate/specified dilutions for appropriate/specified periods of time under appropriate/specified conditions. The blots are washed again with an appropriate washing solution, and the immunoreactive protein bands are visualized, for example, in the case of use of horseradish peroxidase-conjugated anti-immunoglobulin, using enhanced chemiluminescence reagents marketed by Amersham (Bucks, United Kingdom).

To test for a neutralizing effect of antibodies generated in vivo (patients or animals), a relevant biological activity of the pathogen of interest can, for example, be determined by using the bioassays, such, as for example, cell proliferation assays or host adhesion, in varying concentrations of serum of individuals or animals exposed/immunized with the immunogenic polypeptide of interest. Exponentially growing cells of the pathogen are washed and resuspended to a consistent and appropriate concentration in growth medium in a series of serial dilutions, and added in aliquots to each well. For neutralization, a dilution series of serum before and after in vivo exposure (immunization) is added to the wells. The plates are incubated for an appropriate period of time (depending on the pathogen). The growth rate of the pathogen in each well is determined.

A preferred method of screening for immunogenicity is by the Active Maternal Immunization Assay. As discussed in Example 1, this assay may be used to measure serum titers of the female mice during the immunization schedule as well as the survival time of the pups after challenge. The skilled artisan can use the other methods of screening to determine antigenicity or immunogenicity of the immunogenic polypeptides of the present invention set forth in this specification and in the art for screening immunogenic polypeptides.

Methods of screening for antigenicity or immunogenicity may be used to select immunogenic polypeptides of interest from groups of two or more, three or more, five or more, ten or more, or fifty or more immunogenic polypeptides of the present invention based upon a criterion. One of skill in the art may apply any desired criterion in selecting the immunogenic polypeptide of interest. The criterion will depend upon the intended use of the immunogenic polypeptide of interest. By way of example, but not limitation, the criterion may be as simple as selecting the polypeptide with the highest antigenicity or immunogenicity. More complicated criterion may also be used such as selecting the polypeptide with the highest antigenicity or immunogenicity that produces no undesirable side effects upon immunization or selecting a multicomponent vaccine that includes the immunogenic polypeptide that has the highest antigenicity or immunogenicity against a panel of pathogens. Determination of the criterion is a simple matter of experimental design based upon the intended use and therefore one of skill in the art would have no difficulty in selecting appropriate criteria for any situation.

### Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. Antibody includes, for example, vertebrate antibodies, hybrid antibodies, chimeric antibodies, humanized antibodies, altered antibodies, univalent antibodies, Fab proteins, and single domain antibodies.

Antibodies against the proteins of the invention are useful for affinity chromatography, immunoassays, and distinguishing/identifying meningococcal proteins.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25° C. for one hour, followed by incubating at 40° C. for 2-18 hours. The serum is recovered by centrifugation (e.g., 1,000 g for 10 minutes). About 20-50 ml per bleed may be obtained from rabbits.

Monoclonal antibodies are prepared using the standard method of Kohler & Milstein (Nature (1975) 256:495-96), or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen, B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected MAb-secreting hybridomas are then cultured either in vitro (e.g., in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atom s (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the invention.

### Pharmaceutical Compositions

Pharmaceutical compositions can comprise either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the DNA constructs in the individual to which it is administered. Preferred dosages for protein based pharmaceuticals including vaccines will be between 5 and 500 μ5 of the immunogenic polypeptides of the present invention.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutancous applications (e.g., see WO98/20734), needles, and gene guns or hyposprays. Dosage treatment may be a single dose schedule or a multiple dose schedule.

### Vaccines

Vaccines according to the invention may either be prophylactic (i.e., to prevent infection) or therapeutic (i.e., to treat disease after infection).

Such vaccines comprise immunogenic polypeptide(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from such pathogens as diphtheria, tetanus, cholera, H. pylori, etc.

Compositions such as vaccines and pharmaceutical compositions of the invention may advantageously include an adjuvant, which can function to enhance the immune responses (humoral and/or cellular) elicited in a patient who receives the composition.

Adjuvants that can be used with the invention include, but are not limited to:
A mineral containing composition, including calcium salts and aluminum salts (or mixtures thereof). Calcium salts include calcium phosphate (e.g. the “CAP” particles disclosed in ref. 79, which is hereby incorporated by reference for all of its teachings with particular reference to “CAP” particles). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt (Reference 80). Aluminum salt adjuvants are described in more detail below.
Cytokine inducing agents (see in more detail below).
Saponins (chapter 22 of ref. 81), which are a heterologous group of sterol glycosides and triterpenoid glycosides that are found in the bark, leaves, stems, roots and even flowers of a wide range of plant species. Saponin from the bark of the Quillaja saponaria Molina tree have been widely studied as adjuvants. Saponin can also be commercially obtained from Smilax ornata (sarsaparilla), Gypsophila paniculata (brides veil), and Saponaria officinalis (soap root). Saponin adjuvant formulations include purified formulations, such as QS21, as well as lipid formulations, such as ISCOMs. QS21 is marketed as Stimulon™. Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 82 (which is hereby incorporated by reference for all its teachings with particular references to methods of production and use of QS7, QS17, QS18 and QS21). It is possible to use fraction A of Quit A together with at least one other adjuvant (Ref. 83). Saponin formulations may also comprise a sterol, such as cholesterol (84). Combinations of saponins and cholesterols can be used to form unique particles called immunostimulating complexes (ISCOMs) (chapter 23 of ref. 81). ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 85-88 (which is hereby incorporated by reference for all its teachings with particular references to ISCOMs, methods of manufacture of ISCOMs and methods of use of ISCOMs). Optionally, the ISCOMS may be devoid of additional detergent (Ref. 89). It is possible to use a mixture of at least two ISCOM complexes, each complex comprising essentially one saponin fraction, where the complexes are ISCOM complexes or ISCOM matrix complexes (90). A review of the development of saponin based adjuvants can be found in refs. 91 and 92.

Fatty adjuvants (see in more detail below).

Bacterial ADP-ribosylating toxins (e.g. the E. coli heat labile enterotoxin “LT”, cholera toxin “CT”, or pertussis toxin “PT”) and detoxified derivatives thereof, such as the mutant toxins known as LT-K63 and LT R72 (93). The use of detoxified ADP-ribosylating toxins as mucosal adjuvants is described in ref. 94 and as parenteral adjuvants in ref. 95.

Bioadhesives and mucoadhesives, such as esterified hyaluronic acid microspheres (96) or chitosan and its derivatives (97).

Microparticles (i.e. a particle of ˜100 nm to ˜150 μm in diameter, more preferably ˜200 nm to ˜30 μm in diameter, or ˜500 nm to ˜10 μm in diameter) formed from materials that are biodegradable and non toxic (e.g. a poly(α-hydroxy acid), a polyhydroxybutyric acid, a polyorthoester, a polyanhydride, a polycaprolactone, etc.), with poly(lactide co glycolide) being preferred, optionally treated to have a negatively-charged surface (e.g. with SDS) or a positively-charged surface (e.g. with a cationic detergent, such as CTAB).

Liposomes (Chapters 13 & 14 of ref. 81). Examples of liposome formulations suitable for use as adjuvants are described in refs. 98-100.

Oil in water emulsions (see in more detail below).

Polyoxyethylene ethers and polyoxyethylene esters (101). Such formulations further include polyoxyethylene sorbitan ester surfactants in combination with an octoxynol (102) as well as polyoxyethylene alkyl ethers or ester surfactants in combination with at least one additional non-ionic surfactant such as an octoxynol (103). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

Murarnyl peptides, such as N-acetylmuramyl-L-threonyl-D-isoglutamine (“thr-MDP”), N acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-Tdipalmitoyl-sn-glycero-3-hydroxyphosphoryloxy)-ethylamine (“MTP-PE”).

An outer membrane protein proteosome preparation prepared from a first Gram-negative bacterium in combination with a liposaccharide preparation derived from a second Gram negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides. They have been used as adjuvants for influenza vaccines (104).

Methyl inosine 5′-monophosphate (“MIMP”) (105).

A polyhydroxlated pyrrolizidine compound (106), such as one having formula:

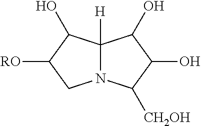

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3 epi casuarine, 7 epi casuarine, 3,7 diepi casuarine, etc.

A gamma inulin (107) or derivative thereof, such as algammulin.

A CD1d ligand, such as a a glycosylceramide e.g. α-galactosylceramide.

These and other adjuvant active substances are discussed in more detail in references 81 & 108.

Compositions may include two or more of said adjuvants. For example, they may advantageously include both an oil in water emulsion and a cytokine inducing agent, as this combination improves the cytokine responses elicited by influenza vaccines, such as the interferon γ response, with the improvement being much greater than seen when either the emulsion or the agent is used on its own.

Antigens and adjuvants in a composition will typically be in admixture.

Where a vaccine includes an adjuvant, it may be prepared extemporaneously, at the time of delivery. Thus the invention provides kits including the antigen and adjuvant components ready for mixing. The kit allows the adjuvant and the antigen to be kept separately until the time of use. The components are physically separate from each other within the kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container. In a preferred arrangement, one of the kit components is in a syringe and the other is in a container such as a vial. The syringe can be used (e.g. with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration. In another preferred arrangement, the two kit components are held together but separately in the same syringe e.g. a dual chamber syringe, such as those disclosed in references 109-116 etc. When the syringe is actuated (e.g. during administration to a patient) then the contents of the two chambers are mixed. This arrangement avoids the need for a separate mixing step at the time of use.

Oil in Water Emulsion Adjuvants

Oil in water emulsions have been found to be particularly suitable for use in adjuvanting influenza virus vaccines. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and may even have a sub micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy(oxy-1,2-ethanediyl) groups, with octoxynol-9 (Triton X 100, or t octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Preferred surfactants for including in the emulsion are Tween 80 (polyoxyethylene sorbitan monooleate), Span 85 (sorbitan trioleate), lecithin and Triton X 100. Mixtures of surfactants can be used e.g. Tween 80/Span 85 mixtures.

Specific oil in water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3%® squalene, 0.5% polysorbate 80 and 0.48% Span 85. This adjuvant is known as ‘MF59’ (117-119), as described in more detail in Chapter 10 of ref. 81 and chapter 12 of ref. 108. The MF59 emulsion advantageously includes citrate ions e.g. 10 mM sodium citrate buffer.
An emulsion of squalene, a tocopherol, and Tween 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably <1 as this provides a more stable emulsion. One such emulsion can be made by dissolving Tween 80 in PBS to give a 2% solution, then mixing 90 ml of this solution with a mixture of (5 g of DL α tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100).
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl MDP in the “SAF 1” adjuvant (120) (0.05-1% Thr MDP, 5% squalane, 2.5% Pluronic L121 and 0.2% polysorbate 80). It can also be used without the Thr MDP, as in the “AF” adjuvant (121) (5% squalane, L25% Pluronic L121 and O₂% polysorbate 80). Microfluidisation is preferred.
An emulsion having from 0.5 50% of an oil, 0.1 10% of a phospholipid, and 0.05 5% of a non ionic surfactant. As described in reference 122, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 123, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles (124).

The emulsions may be mixed with antigen extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

After the antigen and adjuvant have been mixed, the antigen will generally remain in aqueous solution but may distribute itself around the oil/water interface. In general, little if any antigen will enter the oil phase of the emulsion.

Where a composition includes a tocopherol, any of the α, β, γ, δ, ε or ξ tocopherols can be used, but α tocopherols are preferred. The tocopherol can take several forms e.g. different salts and/or isomers. Salts include organic salts, such as succinate, acetate, nicotinate, etc. D a tocopherol and DL a tocopherol can both be used. Tocopherols are advantageously included in vaccines for use in elderly patients (e.g. aged 60 years or older) because vitamin E has been reported to have a positive effect on the immune response in this patient group (125). They also have antioxidant properties that may help to stabilize the emulsions (126). A preferred a tocopherol is DL a tocopherol, and the preferred salt of this tocopherol is the succinate. The succinate salt has been found to cooperate with TNF related ligands in vivo. Moreover, α tocopherol succinate is known to be compatible with vaccines (for example, influenza vaccines) and to be a useful preservative as an alternative to mercurial compounds (127).

### Cytokine-Inducing Agents

Cytokine inducing agents for inclusion in compositions of the invention are able, when administered to a patient, to elicit the immune system to release cytokines, including interferons and interleukins. Cytokine responses are known to be involved in the early and decisive stages of host defense against pathogen infection (128). Preferred agents can elicit the release of one or more of: interferon γ; interleukin 1; interleukin 2; interleukin 12; TNF α; TNF β; and GM CSF. Preferred agents elicit the release of cytokines associated with a Th 1-type immune response e.g. interferon γ, TNF α, interleukin 2. Stimulation of both interferon γ and interleukin 2 is preferred.

As a result of receiving a composition of the invention, therefore, a patient will have T cells that, when stimulated with an antigen, will release the desired cytokine(s) in an antigen specific manner. For example, T cells purified form their blood will release γ interferon when exposed in vitro to the stimulated antigen. Methods for measuring such responses in peripheral blood mononuclear cells (PBMC) are known in the art, and include ELISA, ELISPOT, flow cytometry and real time PCR. For example, reference 129 reports a study in which antigen specific T cell-mediated immune responses against tetanus toxoid, specifically γ interferon responses, were monitored, and found that ELISPOT was the most sensitive method to discriminate antigen specific TT-induced responses from spontaneous responses, but that intracytoplasmic cytokine detection by flow cytometry was the most efficient method to detect re stimulating effects.

Suitable cytokine inducing agents include, but are not limited to:
An immunostimulatory oligonucleotide, such as one containing a CpG motif (a dinucleotide sequence containing an unmethylated cytosine linked by a phosphate bond to a guanosine), or a double stranded RNA, or an oligonucleotide containing a palindromic sequence, or an oligonucleotide containing a poly(dG) sequence.
3 O deacylated monophosphoryl lipid A (‘3dMPL’, also known as ‘MPL™’) (130-133).
An imidazoquinoline compound, such as Imiquimod (“R 837”) (134, 135), Resiquimod (“R 848”) (136), and their analogs; and salts thereof (e.g. the hydrochloride salts). Further details about immunostimulatory imidazoquinolines can be found in references 137 to 141.
A thiosemicarbazone compound, such as those disclosed in reference 142. Methods of formulating, manufacturing, and screening for active compounds are also described in reference 142. The thiosemicarbazones are particularly effective in the stimulation of human peripheral blood mononuclear cells for the production of cytokines, such as TNF-α.
A tryptanthrin compound, such as those disclosed in reference 143. Methods of formulating, manufacturing, and screening for active compounds are also described in reference 143. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

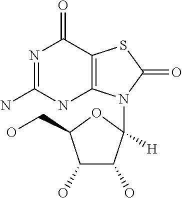

and prodrugs thereof; (b) ANA975; (c) ANA-025-1; (d) ANA380; (e) the compounds disclosed in references 144 to 146; (f) a compound having the formula:

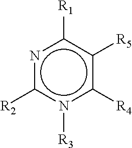

wherein:
R1 and R2 are each independently H, halo, —NRaRb, —OH, C1-6 alkoxy, substituted C1-6 alkoxy, heterocyclyl, substituted heterocyclyl, C6-10 aryl, substituted C6-10 aryl, C1-6 alkyl, or substituted C1-6 alkyl;
R3 is absent, H, C1-6 alkyl, substituted C1-6 alkyl, C6-10 aryl, substituted C6-10 aryl, heterocyclyl, or substituted heterocyclyl;
R4 and R5 are each independently H, halo, heterocyclyl, substituted heterocyclyl, C(O)-Rd, C1-6 alkyl, substituted C1-6 alkyl, or bound together to form a 5 membered ring as in R4-5:

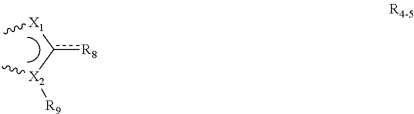

the binding being achieved at the bonds indicated by a [symbol]
X1 and X2 are each independently N, C, O, or S;
R8 is H, halo, —OH, C1-6 alkyl, C2-6 alkenyl, C2-6 alkynyl, —OH, —NRaRb, —(CH2)n-O-Rc, —O—(C1-6 alkyl), —S(O)pRe, or —C(O)-Rd;
R9 is H, C1-6 alkyl, substituted C1-6 alkyl, heterocyclyl, substituted heterocyclyl or R9a, wherein R9a is:

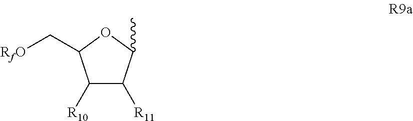

the binding being achieved at the bond indicated by a [symbol]
R10 and R11 are each independently H, halo, C1-6 alkoxy, substituted C1-6 alkoxy, —NRaRb, or —OH;
each Ra and Rv is independently H, C1-6 alkyl, substituted C1-6 alkyl, —C(O)Rd, C6-10 aryl;
each Rc is independently H, phosphate, diphosphate, triphosphate, C1-6 alkyl, or substituted C1-6 alkyl;
each Rd is independently H, halo, C1-6 alkyl, substituted C1-6 alkyl, C1-6 alkoxy, substituted C1-6 alkoxy, —NH2, —NH(C1-6 alkyl), —NH (substituted C1-6 alkyl), —N(C1-6 alkyl)2, —N(substituted C1-6 alkyl)2, C6-10 aryl, or heterocyclyl;
each Re is independently H, C1-6 alkyl, substituted C1-6 alkyl, C6-10 aryl, substituted C6-10 aryl, heterocyclyl, or substituted heterocyclyl;
each Rf is independently H, C1-6 alkyl, substituted C1-6 alkyl, —C(O)Rd, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer.
   Loxoribine (7-allyl-8-oxoguanosine) (147).
   Compounds disclosed in reference 148, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds (149, 150), Hydrapthalamide compounds, Benzophenone compounds, Isoxazole compounds, Sterol compounds, Quinazilinone compounds, Pyrrole compounds (151), Anthraquinone compounds, Quinoxaline compounds, Triazine compounds, Pyrazalopyrimidine compounds, and Benzazole compounds (152).
   Compounds disclosed in reference 153.
   An aminoalkyl glucosaminide phosphate derivative, such as RC 529 (154, 155).
   A phosphazene, such as poly(di(carboxylatophenoxy)phosphazene) (“PCPP”) as described, for example, in references 156 and 157.
   Small molecule immunopotentiators (SMIPs) such as:
N2-methyl-1-(2-methylpropyl)-1H-imidazo[4,5-c]quinoline-2,4-diamine
N2,N2-dimethyl-1-(2-methylpropyl)-1H-imidazo[4,5-c]quinoline-2,4-diamine
N2-ethyl-N2-methyl-1-(2-methylpropyl)-1H-imidazo[4,5-c]quinoline-2,4-diamine
N2-methyl-1-(2-methylpropyl)-N2-propyl-1H-imidazo[4,5-c]quinoline-2,4-diamine
1-(2-methylpropyl)-N2-propyl-1H-imidazo[4,5-c]quinoline-2,4-diamine
N2-butyl-1-(2-methylpropyl)-1H-imidazo[4,5-c]quinoline-2,4-diamine
N2-butyl-N2-methyl-1-(2-methylpropyl)-1H-imidazo[4,5-c]quinoline-2,4-diamine
N2-methyl-1-(2-methylpropyl)-N2-pentyl-1H-imidazo[4,5-c]quinoline-2,4-diamine
N2-methyl-1-(2-methylpropyl)-N2-prop-2-enyl-1H-imidazo[4,5-c]quinoline-2,4-diamine
1-(2-methylpropyl)-2-((phenylmethyl)thio)-1H-imidazo[4,5-c]quinolin-4-amine
1-(2-methylpropyl)-2-(propylthio)-1H-imidazo[4,5-c]quinolin-4-amine
2-((4-amino-1-(2-methylpropyl)-1H-imidazo[4,5-c]quinolin-2-yl)(methyl)amino)ethanol
2-((4-amino-1-(2-methylpropyl)-1H-imidazo[4,5-c]quinolin-2-yl)(methyl)amino)ethyl acetate
4-amino-1-(2-methylpropyl)-1,3-dihydro-2H-imidazo[4,5-c]quinolin-2-one
N2-butyl-1-(2-methylpropyl)-N4,N4-bis(phenylmethyl)-1H-imidazo[4,5-c]quinoline-2,4-diamine
N2-butyl-N2-methyl-1-(2-methylpropyl)-N4,N4-bis(phenylmethyl)-1H-imidazo[4,5-c]quinoline-2,4-diamine
N2-methyl-1-(2-methylpropyl)-N4,N4-bis(phenylmethyl)-1H-imidazo[4,5-c]quinoline-2,4-diamine
N2,N2-dimethyl-1-(2-methylpropyl)-N4,N4-bis(phenylmethyl)-1H-imidazo[4,5-c]quinoline-2,4-diamine
1-{4-amino-2-(methyl (propyl)amino)-1H-imidazo[4,5-c]quinolin-1-yl}-2-methylpropan-2-ol
1-(4-amino-2-(propylamino)-1H-imidazo[4,5-c]quinolin-1-yl)-2-methylpropan-2-ol
N4,N4-dibenzyl-1-(2-methoxy-2-methylpropyl)-N2-propyl-1H-imidazo[4,5-c]quinoline-2,4-diamine.

The cytokine inducing agents for use in the present invention may be modulators and/or agonists of Toll-Like Receptors (TLR). For example, they may be agonists of one or more of the human TLR1, TLR2, TLR3, TLR4, TLR7, TLR8, and/or TLR9 proteins. Preferred agents are agonists of TLR7 (e.g. imidazoquinolines) and/or TLR9 (e.g. CpG oligonucleotides). These agents are useful for activating innate immunity pathways.

The cytokine inducing agent can be added to the composition at various stages during its production. For example, it may be within an antigen composition, and this mixture can then be added to an oil in water emulsion. As an alternative, it may be within an oil in water emulsion, in which case the agent can either be added to the emulsion components before emulsification, or it can be added to the emulsion after emulsification. Similarly, the agent may be coacervated within the emulsion droplets. The location and distribution of the cytokine inducing agent within the final composition will depend on its hydrophilic/lipophilic properties e.g. the agent can be located in the aqueous phase, in the oil phase, and/or at the oil water interface.

The cytokine inducing agent can be conjugated to a separate agent, such as an antigen (e.g. CRM197). A general review of conjugation techniques for small molecules is provided in ref. 158. As an alternative, the adjuvants may be non-covalently associated with additional agents, such as by way of hydrophobic or ionic interactions.

Two preferred cytokine inducing agents are (a) immunostimulatory oligonucleotides and (b) 3dMPL.

Immunostimulatory oligonucleotides can include nucleotide modifications/analogs such as phosphorothioate modifications and can be double-stranded or (except for RNA) single-stranded. References 159, 160 and 161 disclose possible analog substitutions e.g. replacement of guanosine with 2′-deoxy-7-deazaguanosine. The adjuvant effect of CpG oligonucleotides is further discussed in refs. 162-167. A CpG sequence may be directed to TLR9, such as the motif GTCGTT or TTCGTT (168). The CpG sequence may be specific for inducing a Th1 immune response, such as a CpG-A ODN (oligodeoxynucleotide), or it may be more specific for inducing a B cell response, such a CpG-B ODN. CpG-A and CpG-B ODNs are discussed in refs. 169-171. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 168 and 172-174. A useful CpG adjuvant is CpG7909, also known as ProMunem (Coley Pharmaceutical Group, Inc.).

As an alternative, or in addition, to using CpG sequences, TpG sequences can be used (175). These oligonucleotides may be free from unmethylated CpG motifs.

The immunostimulatory oligonucleotide may be pyrimidine rich. For example, it may comprise more than one consecutive thymidine nucleotide (e.g. TTTT, as disclosed in ref. 175), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref. 148), and/or it may have a nucleotide composition with >25% cytosine (e.g. >35%, >40%, >50%, >60%, >80%, etc.). These oligonucleotides may be free from unmethylated CpG motifs.

Immunostimulatory oligonucleotides will typically comprise at least 20 nucleotides. They may comprise fewer than 100 nucleotides.

3dMPL (also known as 3 de-O-acylated monophosphoryl lipid A or 3 O desacyl 4′monophosphoryl lipid A) is an adjuvant in which position 3 of the reducing end glucosamine in monophosphoryl lipid A has been de-acylated. 3dMPL has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL 1, IL-12, TNF α and GM-CSF (see also ref. 176). Preparation of 3dMPL was originally described in reference 177.

3dMPL can take the form of a mixture of related molecules, varying by their acylation (e.g. having 3, 4, 5 or 6 acyl chains, which may be of different lengths). The two glucosamine (also known as 2 deoxy-2-amino glucose) monosaccharides are N acylated at their 2 position carbons (i.e. at positions 2 and 2′), and there is also O acylation at the 3′ position. The group attached to carbon 2 has formula —NH—CO—CH2-CR1R1′. The group attached to carbon 2′ has formula —NH—CO—CH2-CR2R2′. The group attached to carbon 3′ has formula —O—CO—CH2-CR3R3′. A representative structure is:

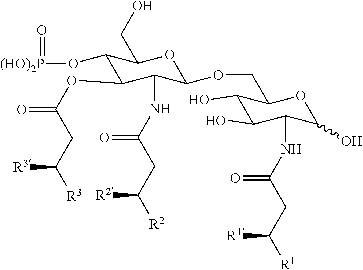

Groups R1, R2 and R3 are each independently —(CH2)n-CH3. The value of n is preferably between 8 and 16, more preferably between 9 and 12, and is most preferably 10.

Groups R1′, R2′ and R3′ can each independently be: (a) —H; (b) —OH; or (c) —O CO R4, where R4 is either —H or —(CH2)m-CH3, wherein the value of m is preferably between 8 and 16, and is more preferably 10, 12 or 14. At the 2 position, m is preferably 14. At the 2′ position, m is preferably 10. At the 3′ position, m is preferably 12. Groups R1′, R2′ and R3′ are thus preferably —O acyl groups from dodecanoic acid, tetradecanoic acid or hexadecanoic acid.

When all of R1′, R2′ and R3′ are —H then the 3dMPL has only 3 acyl chains (one on each of positions 2, 2′ and 3′). When only two of R1′, R2′ and R3′ are —H then the 3dMPL can have 4 acyl chains. When only one of R1′, R2′ and R3′ is —H then the 3dMPL can have 5 acyl chains. When none of R1′, R2′ and R3′ is —H then the 3dMPL can have 6 acyl chains. The 3dMPL adjuvant used according to the invention can be a mixture of these forms, with from 3 to 6 acyl chains, but it is preferred to include 3dMPL with 6 acyl chains in the mixture, and in particular to ensure that the hexaacyl chain form makes up at least 10% by weight of the total 3dMPL e.g. >20%, >30%, >40%, >50% or more. 3dMPL with 6 acyl chains has been found to be the most adjuvant active form.

Thus the most preferred form of 3dMPL for inclusion in compositions of the invention is:

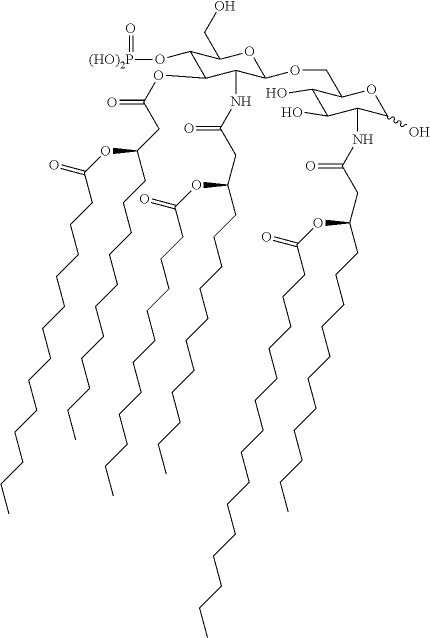

Where 3dMPL is used in the form of a mixture then references to amounts or concentrations of 3dMPL in compositions of the invention refer to the combined 3dMPL species in the mixture.

In aqueous conditions, 3dMPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3dMPL) are preferred for use according to the invention because of their superior activity (178). Preferred particles have a mean diameter less than 220 nm, more preferably less than 200 nm or less than 150 nm or less than 120 nm, and can even have a mean diameter less than 100 nm. In most cases, however, the mean diameter will not be lower than 50 nm. These particles are small enough to be suitable for filter sterilization. Particle diameter can be assessed by the routine technique of dynamic light scattering, which reveals a mean particle diameter. Where a particle is said to have a diameter of x nm, there will generally be a distribution of particles about this mean, but at least 50% by number (e.g. >60%, >70%, >80%, >90%, or more) of the particles will have a diameter within the range x+25%.

3dMPL can advantageously be used in combination with an oil in water emulsion. Substantially all of the 3dMPL may be located in the aqueous phase of the emulsion.

The 3dMPL can be used on its own, or in combination with one or more further compounds. For example, it is known to use 3dMPL in combination with the QS21 saponin (179) (including in an oil in water emulsion (180)), with an immunostimulatory oligonucleotide, with both QS21 and an immunostimulatory oligonucleotide, with aluminum phosphate (181), with aluminum hydroxide (182), or with both aluminum phosphate and aluminum hydroxide.

Fatty Adjuvants

Fatty adjuvants that can be used with the invention include the oil in water emulsions described above, and also include, for example:

A compound of formula I, II or III, or a salt thereof:

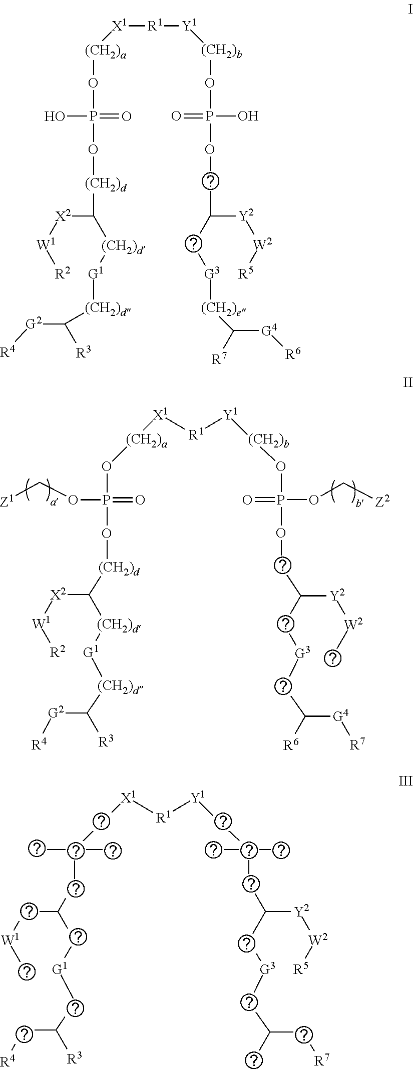

as defined in reference 183, such as ‘ER 803058’, ‘ER 803732’, ‘ER 804053’, ER 804058′, ‘ER 804059’, ‘ER 804442’, ‘ER 804680’, ‘ER 804764’, ER 803022 or ‘ER 804057’ e.g.:

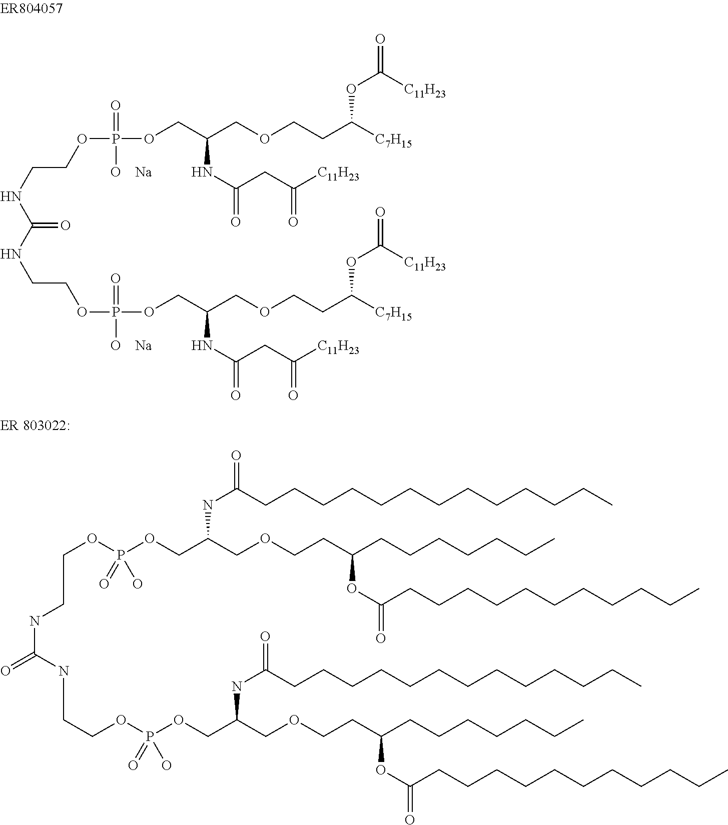

Derivatives of lipid A from Escherichia coli such as OM-174 (described in refs. 184 and 185).

A formulation of a cationic lipid and a (usually neutral) co-lipid, such as aminopropyl-dimethyl-myristoleyloxy-propanaminium bromide-diphytanoylphosphatidyl-ethanolamine (“Vaxfectin™”) or aminopropyl-dimethyl-bis-dodecyloxy-propanaminium bromide-dioleoylphosphatidyl-ethanolamine (“GAP-DLRIE:DOPE”). Formulations containing (+)-N-(3-aminopropyl)-N,N-dimethyl-2,3-bis(syn-9-tetradeceneyloxy)-1-propanaminium salts are preferred (186).

3 O deacylated monophosphoryl lipid A (see above).

Compounds containing lipids linked to a phosphate-containing acyclic backbone, such as the TLR4 antagonist E5564 (187, 188):

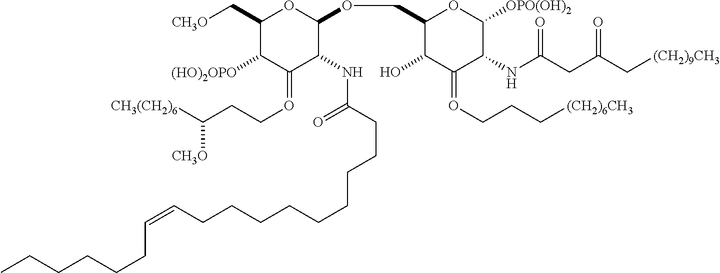

Aluminum Salt Adjuvants

The adjuvants known as aluminum hydroxide and aluminum phosphate may be used. These names are conventional, but are used for convenience only, as neither is a precise description of the actual chemical compound which is present (e.g. see chapter 9 of reference 81). The invention can use any of the “hydroxide” or “phosphate” adjuvants that are in general use as adjuvants.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 81). The degree of crystallinity of an aluminium hydroxide adjuvant is reflected by the width of the diffraction band at half height (WHH), with poorly crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AIPO₄ by the presence of hydroxyl groups. For example, an IR spectrum band at 3164 cm⁻¹ (e.g. when heated to 200° C.) indicates the presence of structural hydroxyls (ch. 9 of ref. 81).

The PO₄/Al³⁺ molar ratio of an aluminium phosphate adjuvant will generally be between 0.3 and 1.2, preferably between 0.8 and 1.2, and more preferably 0.95+0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate like morphology as seen in transmission electron micrographs). Typical diameters of the particles are in the range 0.5-20 μm (e.g. about 5 10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate (77). In this case there may be more aluminium phosphate than hydroxide e.g. a weight ratio of at least 2:1 e.g. >5:1, >6:1, >7:1, >8:1, >9:1, etc.

The concentration of Al⁺⁺⁺ in a composition for administration to a patient is preferably less than 10 mg/ml e.g. <5 mg/ml, <4 mg/ml, <3 mg/ml, <2 mg/ml, <1 mg/ml, etc. A preferred range is between 0.3 and 1 mg/ml.

As well as including one or more aluminium salt adjuvants, the adjuvant component may include one or more further adjuvant or immunostimulating agents. Such additional components include, but are not limited to: a 3-O-deacylated monophosphoryl lipid A adjuvant (‘3d MPL’); and/or an oil in water emulsion. 3d MPL has also been referred to as 3 de-O-acylated monophosphoryl lipid A or as 3 O desacyl 4′ monophosphoryl lipid A. The name indicates that position 3 of the reducing end glucosamine in monophosphoryl lipid A is de-acylated. It has been prepared from a heptoseless mutant of S. minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF α and GM-CSF. Preparation of 3d MPL was originally described in reference 150, and the product has been manufactured and sold by Corixa Corporation under the name MPL™. Further details can be found in refs 130 to 133.

Vaccines produced by the invention may be administered to patients at substantially the same time as (e.g. during the same medical consultation or visit to a healthcare professional) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a pneumococcal conjugate vaccine, etc. Administration at substantially the same time as a pneumococcal vaccine is particularly useful in elderly patients.

The composition may include an antibiotic.

Immunogenic compositions used as vaccines comprise an immunologically effective amount of the immunogenic polypeptide or immunogenic polypeptides, as well as any other of the above-mentioned components, as needed. By “immunologically effective amount,” it is meant that the administration of that amount to an individual, either in a single dose or as part of a series, is effective for treatment or prevention. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, e.g., by injection, either subcutaneously, intramuscularly, or transdermally/transcutaneously (e.g., WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents, As an alternative to protein-based vaccines, DNA vaccination may be employed (e.g., Robinson & Torres (1997) Seminars in Immunology 9:271-283; Donnelly et al. (1997) Annu Rev Immunol 15:617-648; see later herein).

### Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in vivo or ex vivo. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153. Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses e.g., MCF and MCF-MLV (see Kelly (1983) J. Virol. 45:291), spurnaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g., HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC No. VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curie (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, in most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e., there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector, Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar Institute), pHSVIac described in Geller (1988) Science 241:1667-1669 and in WO90/09441 and WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with the ATCC as accession numbers ATCC VR-977 and ATCC VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679,640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1 110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1 244; Ndumu virus, for example ATCC VR-37 1; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curie&rsqb; (1992) Hum Gene Ther 3:147-154 ligand linked DNA, for example see Wu (1989) J Biol Chem 264:16985-16987, eukaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/1 1033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin, Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033. Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W.H. Freeman, San Francisco; Szoka (1980) Biochem Biophys Acta 600:1; Bayer (1979) Biochem BiophysActa 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

### Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g., see WO98/20734), needles, and gene guns or hyposprays. Dosage treatment may be a single dose schedule or a multiple dose schedule.

Methods for the ex vivo delivery and reimplantation of transformed cells into a subject are known in the aft and described in e.g., WO93/14778. Examples of cells useful in ex vivo applications include, for example, stem cells, particularly hematopoietic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

### Polynucleotide and Polypeptide Pharmaceutical Compositions

In addition to the pharmaceutically acceptable carriers and salts described above, the following additional agents can be used with polynucleotide and/or polypeptide compositions.

i. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII

ii. Hormones, Vitamins, etc.

Other groups that can be included are, for example: hormones, steroids, androgens, estrogens, thyroid hormone, or vitamins, folic acid.

iii. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

iv. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Feigner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N(1-2,3-dioleyloxy)propyl)-N,N,N-triethylammonium (DOTMA) liposomes are available under the trademark Lipofectin, from GIBCO BRL, Grand Island, N.Y. (See, also, Feigner supra). Other commercially available liposomes include transfectace (DDAB/DOPE) and DOTAP/DOPE (Boerhinger). Other cationic liposomes can be prepared from readily available materials using techniques well known in the art. See, e.g., Szoka (1978) Proc. Natl. Acad. Sci. USA 75:4194-4198; WO90/11092 for a description of the synthesis of DOTAP (1 2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g., Straubinger (1983) Meth. Immunol. 101:512-527; Szoka (1978) Proc. Nall. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

v. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, Al, All, AIV; CI, CII, CIII.

A lipoprotein can comprise more than one apoprotein. For example, naturally occurring chylomicrons comprises of A, B, C, and E, over time these lipoproteins lose A and acquire C and E apoproteins. VLDL comprises A, B, C, and E apoproteins, LDL comprises apoprotein B; and HDL comprises apoproteins A, C, and E. The amino acid of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet. 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J. Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim BiophysActa 30:443. Lipoproteinscan also be purchased from commercial suppliers, such as Biomedical Technologies, Inc., Stoughton, Mass., USA. Further description of lipoproteins can be found in Zuckermann et. al. WO98/06437.

vi. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc. The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic acid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and putrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

### Immunodiagnostic Assays

Another aspect of the present invention includes GBS 80 immunogenic polypeptides of the present invention used in immunoassays to detect antibody levels (or, conversely, anti-Streptococcal antibodies can be used to detect antigen levels). Immunoassays based on well defined, recombinant antigens can be developed to replace invasive diagnostics methods. Antibodies to GBS 80 immunogenic polypeptides within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

### Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. (supra) Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 12° to 20° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tm=81+16.6(log Ci)+0.4(%(G+C))−0.6(% formamide)−600/n−1.5(% mismatch).

where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e., stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and re-exposed. If the time required for exposure makes this approach impractical, several hybridization and/or washing stringencies should be tested in parallel.

### Combinations Including GBS 80

Another aspect of the present invention includes combination of one or more of the immunogenic polypeptides with other GBS antigens. Preferably, the combination of GBS antigens consists of three, four, five, six, seven, eight, nine, or ten GBS antigens. Still more preferably, the combination of GBS antigens consists of three, four, or five GBS antigens. Such combinations may include full length and/or antigenic fragments of the respective antigens and include combinations where the polypeptides and antigens are physically linked to one another and combinations where the polypeptides and antigens are not physically linked but are included in the same composition.

Preferably, the combinations of the invention provide for improved immunogenicity over the immunogenicity of the antigens when administered alone. Improved immunogenicity may be measured, for example, by the Active Maternal Immunization Assay. As discussed in Example 1, this assay may be used to measure serum titers of the female mice during the immunization schedule as well as the survival time of the pups after challenge. Preferably, immunization with the immunogenic compositions of the invention yield an increase of at least 2 percentage points (preferably at least 3, 4 or 5 percentage points) in the percent survival of the challenged pups as compared to the percent survival from maternal immunization with a single antigen of the composition when administered alone. Preferably, the increase is at least 6, 7, 8, 9, 10, 11, 12, 13, 14, 15, 16, 17, 18, 19, 20, 21, 22, 23, 24, 25, 26, 27, 28, 29 or 30 percentage points. Preferably, the GBS combinations of the invention comprising GBS 80 demonstrate an increase in the percent survival as compared to the percent survival from immunization with a non-GBS 80 antigen alone.

According to one embodiment of the invention, combinations of antigens or fusion proteins containing a portion or portions of the antigens will include GBS 80 or a portion thereof in combination with from one to 10 antigens, preferably one to 10 or less antigens. Examples of GBS antigens may be found in U.S. Ser. No. 10/415,182, filed Apr. 28, 2003, the International Applications (WO04/041157 and WO05/028618), and WO04/099242, each of which is hereby incorporated in its entirety.

### GBS Polysaccharides

The compositions of the invention may be further improved by including GBS polysaccharides. Preferably, the GBS antigen and the saccharide each contribute to the immunological response in a recipient. The combination is particularly advantageous where the saccharide and polypeptide provide protection from different GBS serotypes.

The combined antigens may be present as a simple combination where separate saccharide and polypeptide antigens are administered together, or they may be present as a conjugated combination, where the saccharide and polypeptide antigens are covalently linked to each other.

Thus the invention provides an immunogenic composition comprising (i) one or more GBS polypeptide antigens and (ii) one or more GBS saccharide antigens. The polypeptide and the polysaccharide may advantageously be covalently linked to each other to form a conjugate.

Between them, the combined polypeptide and saccharide antigens preferably cover (or provide protection from) two or more GBS serotypes (e.g. 2, 3, 4, 5, 6, 7, 8 or more serotypes). The serotypes of the polypeptide and saccharide antigens may or may not overlap. For example, the polypeptide might protect against serogroup II or V, while the saccharide protects against either serogroups Ia, Ib, or III. Preferred combinations protect against the following groups of serotypes: (1) serotypes Ia and Ib, (2) serotypes Ia and II, (3) serotypes Ia and III, (4) serotypes Ia and IV, (5) serotypes Ia and V, (6) serotypes Ia and VI, (7) serotypes Ia and VII, (8) serotypes Ia and VIII, (9) serotypes Ib and II, (10) serotypes Ib and III, (11) serotypes Ib and IV, (12) serotypes Ib and V, (13) serotypes Ib and VI, (14) serotypes Ib and VII, (15) serotypes Ib and VIII, 16) serotypes II and m, (17) serotypes II and IV, (18) serotypes II and V, (19) serotypes II and VI, (20) serotypes II and III, (21) serotypes II and VII, (22) serotypes III and IV, (23) serotypes III and V, (24) serotypes III and VI, (25) serotypes III and VII, (26) serotypes III and VIII, (27) serotypes IV and V, (28) serotypes IV and VI, (29) serotypes IV and VII, (30) serotypes IV and VIII, (31) serotypes V and VI, (32) serotypes V and VII, (33) serotypes V and VIII, (34) serotypes VI and VII, (35) serotypes VI and VIII, and (36) serotypes VII and VIII.

Still more preferably, the combinations protect against the following groups of serotypes: (1) serotypes Ia and II, (2) serotypes Ia and V, (3) serotypes Ib and II, (4) serotypes Ib and V, (5) serotypes III and II, and (6) serotypes III and V. Most preferably, the combinations protect against serotypes III and V. Protection against serotypes II and V is preferably provided by polypeptide antigens.

Protection against serotypes Ia, Ib and/or III may be polypeptide or saccharide antigens.

In one embodiment, the immunogenic composition comprises a GBS saccharide antigen and at least two GBS polypeptide antigens or fragments thereof, wherein said GBS saccharide antigen comprises a saccharide selected from GBS serotype Ia, Ib, and III, and wherein said GBS polypeptide antigens comprise a combination of at least two polypeptide or a fragment thereof selected from the antigen group consisting of GBS 80 (gi:2253618), GBS 67 (gi22537555), SAN1518 (Spbl, gi:77408651), GBS104 and GBS 322 (the foregoing antigens are described in U.S. patent application Ser. No. 11/192,046, which is hereby incorporated by reference for all that it teaches and in particular for the antigens and fragments thereof). Preferably, the combination includes GBS 80 or a fragment thereof.

### Further Antigens

The compositions of the invention may further comprise one or more additional non-GBS antigens, including additional bacterial, viral or parasitic antigens.

In another embodiment, the GBS antigen combinations of the invention are combined with one or more additional, non-GBS antigens suitable for use in a vaccine designed to protect elderly or immunocomprised individuals. For example, the GBS antigen combinations may be combined with an antigen derived from the group consisting of Enterococcus faecalis, Staphylococcus aureus, Staphylococcus epidermis, Pseudomonas aeruginosa, Legionella pneumophila, Listeia monocytogenes, Neisseria meningitides, influenza, and Parainfluenza virus (‘PIV’).

Where a saccharide or carbohydrate antigen is used, it is preferably conjugated to a carrier protein in order to enhance immunogenicity {e.g. refs. 42 to 51}. Preferred carrier proteins are bacterial toxins or toxoids, such as diphtheria or tetanus toxoids. The CRM97 diphtheria toxoid is particularly preferred {52}. Other carrier polypeptides include the N. meningitidis outer membrane protein {53}, synthetic peptides {54, 55}, heat shock proteins {56, 57}, pertussis proteins (58, 59), protein D from H. influenzae {60}, cytokines 61), lymphokines, hormones, growth factors, toxin A or B from C. difficile {62}, iron uptake proteins (63), etc. Where a mixture comprises capsular saccharides from both serogroups A and C, it may be preferred that the ratio (w/w) of MenA saccharide:MenC saccharine is greater than 1 (e.g. 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used {e.g. refs. 64 to 72}. Protein components of the compositions of the invention may thus be replaced by nucleic acid (preferably DNA e.g. in the form of a plasmid) that encodes the protein.

### EXAMPLES

### Example 1

As described in WO05/028618, both an Active Maternal Immunization Assay and a Passive Maternal Immunization Assay were conducted on fragments of the GBS 80 protein.

As used herein, an Active Maternal Immunization assay refers to an in vivo protection assay where female mice are immunized with the test antigen composition. The female mice are then bred and their pups are challenged with a lethal dose of GBS. Serum titers of the female mice during the immunization schedule are measured as well as the survival time of the pups after challenge.

Specifically, the Active Maternal Immunization assays referred to herein used groups of four CD-1 female mice (Charles River Laboratories, Calco Italy). These mice were immunized intraperitoneally with the selected proteins in Freund's adjuvant at days 1, 21 and 35, prior to breeding. 6-8 weeks old mice received 20 μg protein/dose when immunized with a single antigen, 30-45 μg protein/dose (15 μg each antigen) when immunized with combination of antigens. The immune response of the dams was monitored by using serum samples taken on day 0 and 49. The female mice were bred 2-7 days after the last immunization (at approximately t=36-37), and typically had a gestation period of 21 days. Within 48 hours of birth, the pups were challenged via I.P. with GBS in a dose approximately equal to an amount which would be sufficient to kill 70-90% of unimmunized pups (as determined by empirical data gathered from PBS control groups). The GBS challenge dose is preferably administered in 50 μl of THB medium. Preferably, the pup challenge takes place at 56 to 61 days after the first immunization. The challenge inocula were prepared starting from frozen cultures diluted to the appropriate concentration with THB prior to use. Survival of pups was monitored for 5 days after challenge.

As used herein, the Passive Maternal Immunization Assay refers to an in vivo protection assay where pregnant mice are passively immunized by injecting rabbit immune sera (or control sera) approximately 2 days before delivery. The pups are then challenged with a lethal dose of GBS.

Specifically, the Passive Maternal Immunization Assay referred to herein used groups of pregnant CD1 mice which were passively immunized by injecting 1 ml of rabbit immune sera or control sera via I.P., 2 days before delivery. Newborn mice (24-48 hrs after birth) are challenged via I.P. with a 70-90% lethal dose of GBS serotype III COH1. The challenge dose, obtained by diluting a frozen mid log phase culture, was administered in 500 of THB medium.

For both assays, the number of pups surviving GBS infection was assessed every 12 hrs for 4 days. Statistical significance was estimated by Fisher's exact test.

The results of each assay for immunization with SEQ ID NO: 5, SEQ ID NO: 6, PBS and GBS whole cell are set forth in Tables 1 and 2 below.

TABLE 1
Active Maternal Immunization

| | | % | |
|---|---|---|---|
| Antigen | Alive/total | Survival | Fisher's exact test |
| PBS (neg control) | 13/80 | 16% | |
| GBS (whole cell) | 54/65 | 83% | P < 0.00000001 |
| GBS 80 (intact) | 62/70 | 88% | P < 0.0000001 |
| GBS 80 (fragment) SEQ ID5 | 35/64 | 55% | P = 0.0000013 |
| GBS 80 (fragment) SEQ ID6 | 13/67 | 19% | P = 0.66 |

TABLE 2
Passive Maternal Immunization

| | | % | |
|---|---|---|---|
| Antigen | Alive/total | Survival | Fisher's exact test |
| PBS (neg control) | 12/42 | 28% | |
| GBS (whole cell) | 48/52 | 92% | P < 0.0000001 |
| GBS 80 (intact) | 48/55 | 87% | P < 0.00000001 |
| GBS 80 (fragment) SEQ ID5 | 45/57 | 79% | P = 0.0000006 |
| GBS 80 (fragment) SEQ ID6 | 13/54 | 24% | P = 1 |

As shown in Tables 1 and 2, immunization with the SEQ ID NO: 5 GBS 80 fragment provided a substantially improved survival rate for the challenged pups than the comparison SEQ ID NO: 6 GBS 80 fragment. These results indicate that the SEQ ID NO: 5 GBS 80 fragment contains an important immunogenic epitope of GBS 80.

### Example 2

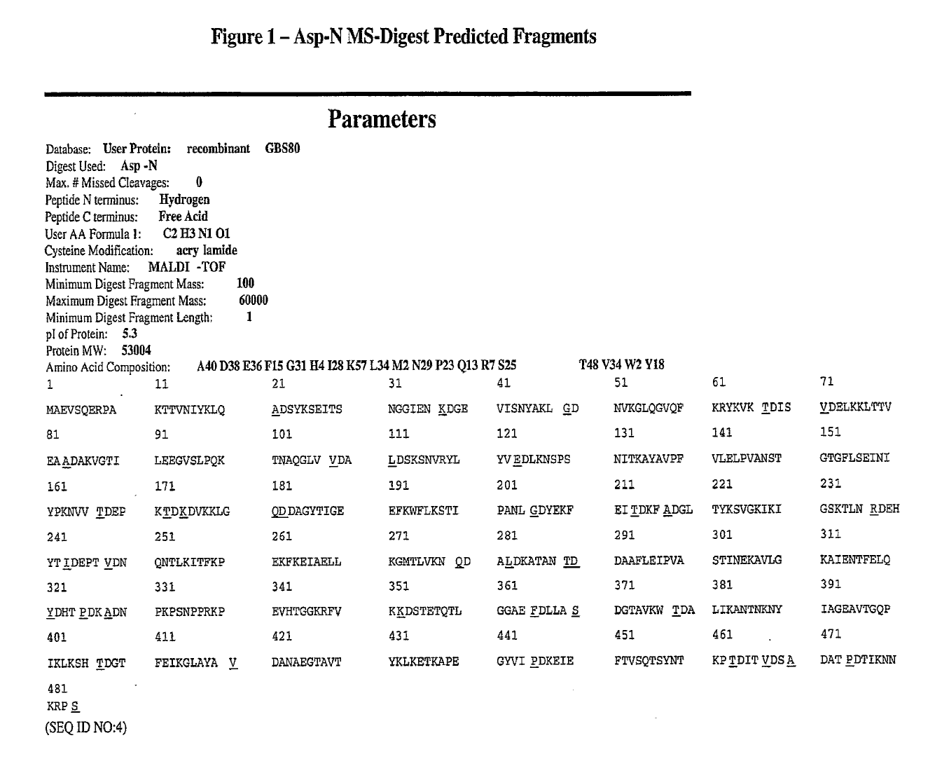

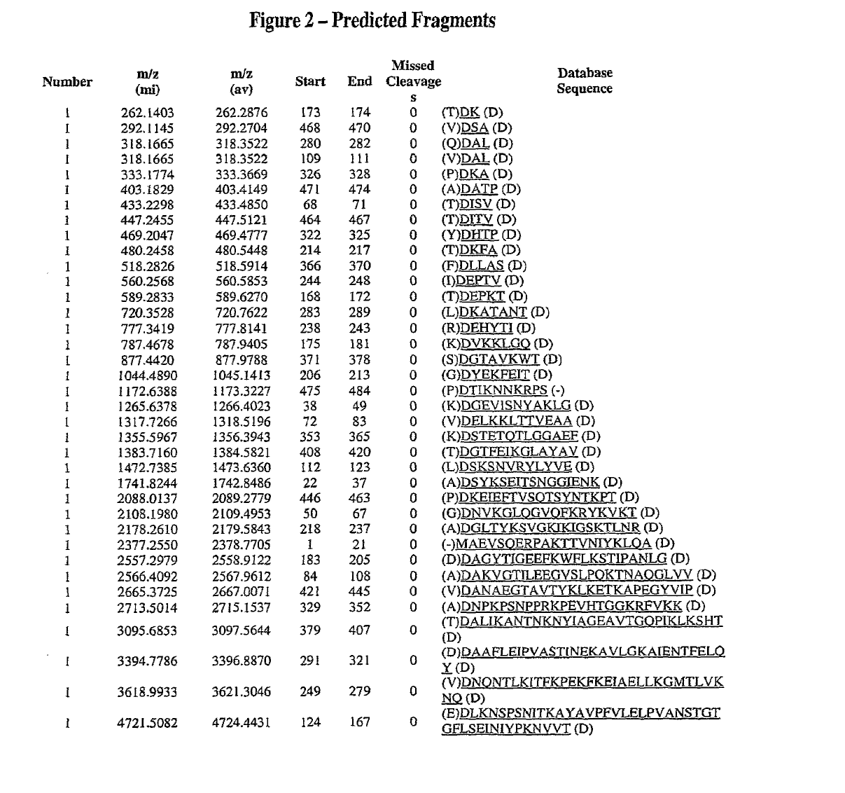

Epitope mapping was conducted to identify the immunogenic polypeptides of the present invention. First, GBS 80 was subject to total digestion with the Asp-N. FIGS. 1 and 2 show the predicted fragments and their size on MALDI-TOF. Representative conditions for total digestion with Asp-N were:
Add 0.1% RapiGest SF (Waters) to 100 μl of GBS80 lot F (1.7 μg/μl) and heat at 98° C. for 7 minutes.
Add 2 μg of Endoproteinase Asp-N (Roche) reconstituted in 5 μl double distilled water.
Incubate at 37° C. for 2 hours.
Add 0.2% formic acid to stop the digestion.
Store at −20° C.
   After total digestion, the peptides were separated by reverse phase chromatography. The identity of each purified peptide was assessed by MALDI-TOF

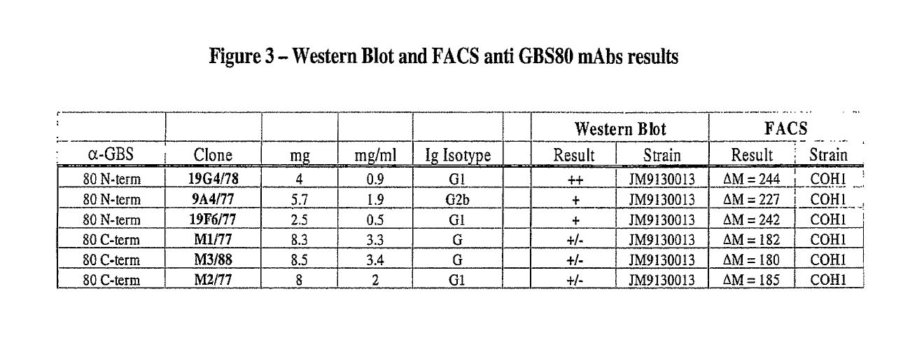

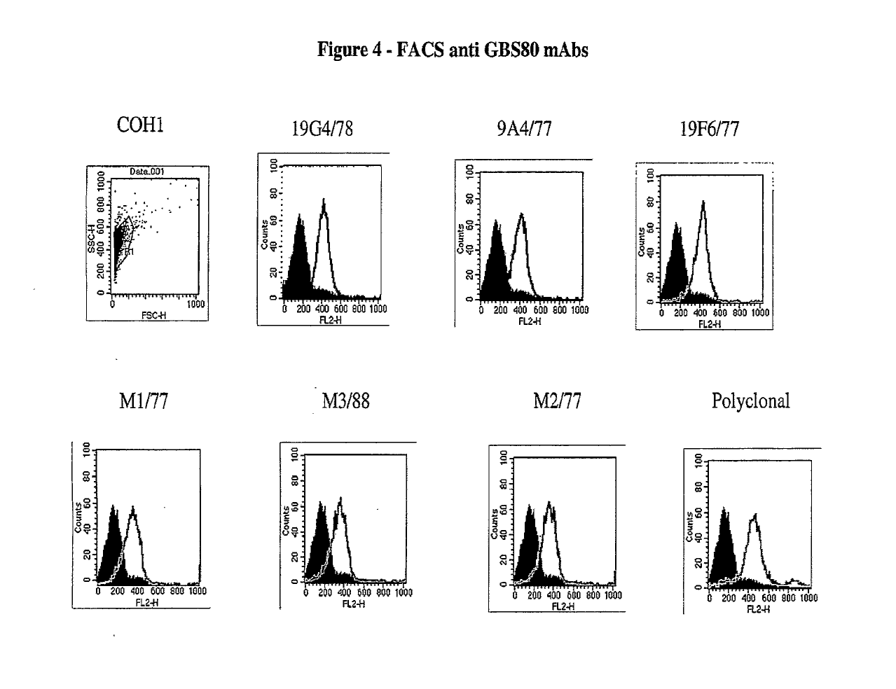

The specific immunogenic polypeptides were identified by mapping epitopes found within the GBS 80 protein using six different mouse monoclonal antibodies that specifically bind to the GBS 80 protein. Three monoclonal antibodies were identified from a pool of Hybridoma generated by immunizing a mouse with full-length GBS 80: 9A4/77, 19G4178, and 19F6177. Three additional antibodies were identified from a pool of Hybridoma generated by immunizing a mouse also with full-length GBS 80: M3/88, M1/77, and M2/77. FIGS. 3 and 4 summarize the results of FACs analysis and western blots with the six monoclonal antibodies.

The GBS 80 protein was subject to partial digestion with either Asp-N or Arg-C. Representative conditions for partial digestion with Asp-N were as described above. Representative conditions for partial digestion with Arg-C were:
Added 0.1% RapiGest SF (Waters) to 100 μl of GBS80 lot F (1.7 μg/μl) and to 100 μl of GBS80 lot 3 (2 μg/μl) and heated at 98° C. for 7 minutes.
Added 2.5 μg of Endoproteinase Arg-C (Roche) to each sample reconstituted in 25 μl double distilled water.
Incubated at 37° C. for 2 hours.
Added 0.2% formic acid to stop the digestion.
Stored at −20° C.

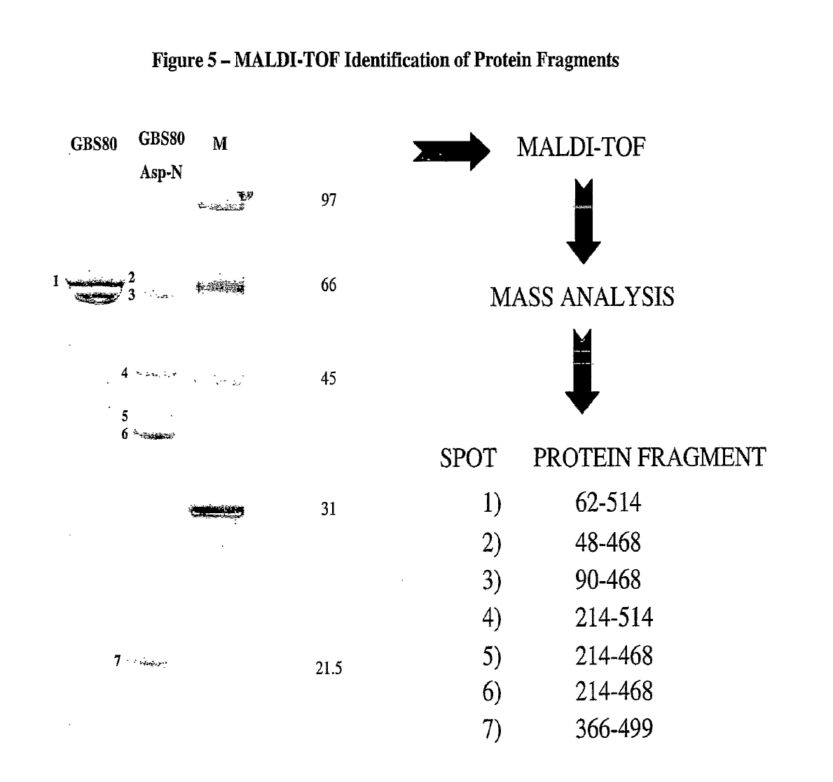

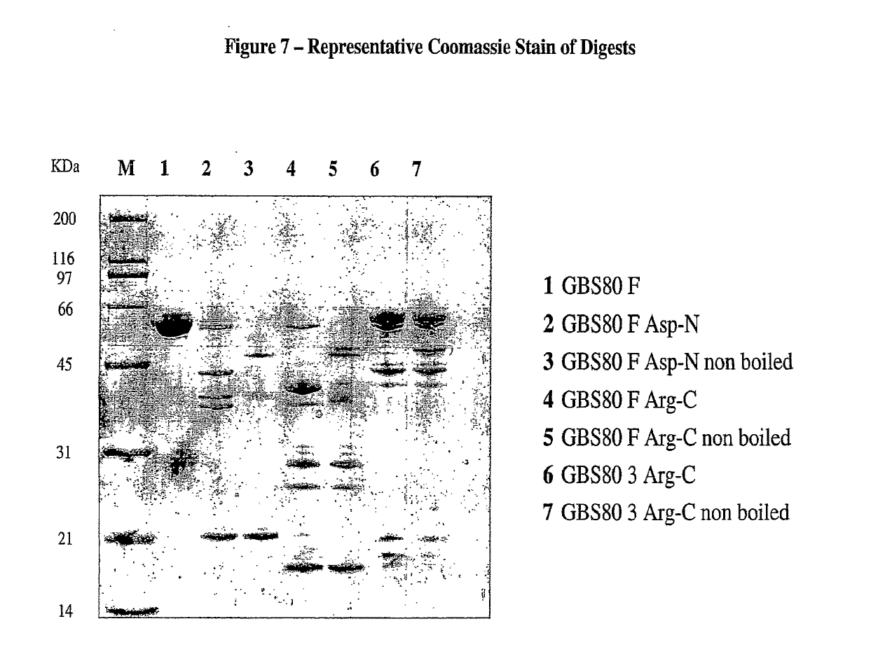

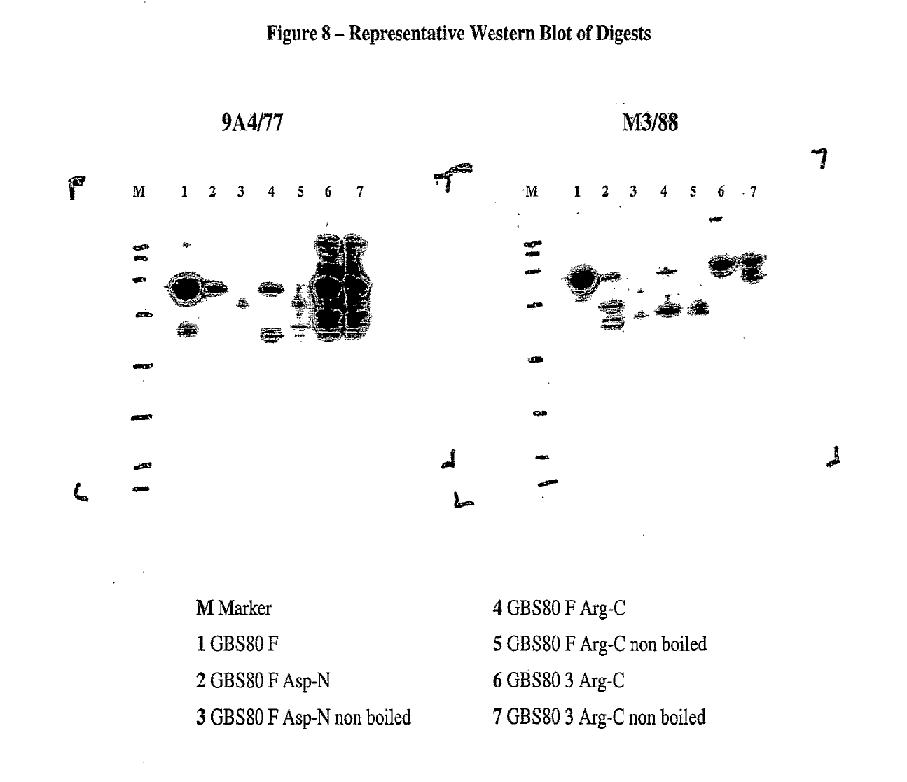

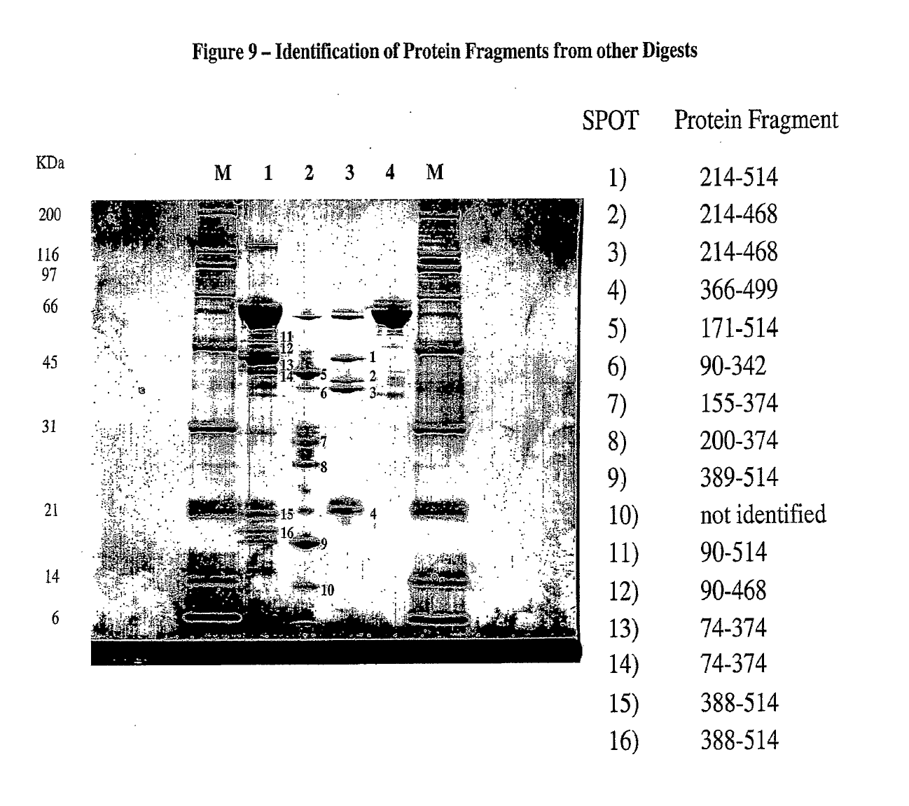

The partial digests were then run on SDS PAGE. The gels were stained with Coomassie Blue and the individual bands were isolated and subject to MALDI-TOF to determine the identity of each band on the gel (See e.g., FIG. 5 showing an example of identification of the bands produced by partial digestion of GBS 80 with Asp-N).
Excise bands of interest from the acrylamide gel and transfer to clean Eppendorf tubes.
Add 100 μl of destaining solution (50% acetonitrile/50 mM ammonium bicarbonate) and allow the gel pieces to detain by shaking the tubes.
Remove destaining solution and wash with 20 μl of acetonitrile.
Dry the gel pieces.
Cover the gel pieces with 12 μl of digestion solution (10 μg/ml Promega Trypsin in 50 mM ammonium bicarbonate).
Incubate at 37° C. for 2 hours.
Transfer the digestion solution to clean Eppendorf tubes and add 5 μl of 0.1% TFA.
Purify the tryptic peptides with MAP and analyze with MALDI-TOF.
   FIG. 7 shows an SDS PAGE gel stained with Coomassie blue comparing partial digests of GBS 80 with and without boiling to denature GBS 80 and two different proteases. GBS 80 F and GBS 80 3 represent different conformer of GBS 80 which may be purified from one another and have different protease sensitivities as shown in FIG. 7. FIG. 8 shows a representative western blot of the SDS PAGE gel shown in FIG. 7. As expected, the monoclonal antibody generated with the N-terminal portion of GBS 80 shows a distinct pattern as compared to the monoclonal antibody generated with the C-terminal portion of GBS 80. FIG. 9 shows an SDS PAGE gel of the partial digest produced from boiled samples of OBS 80 with the identity of the protein fragments on the right side of the figure as determined with MALDI-TOF.

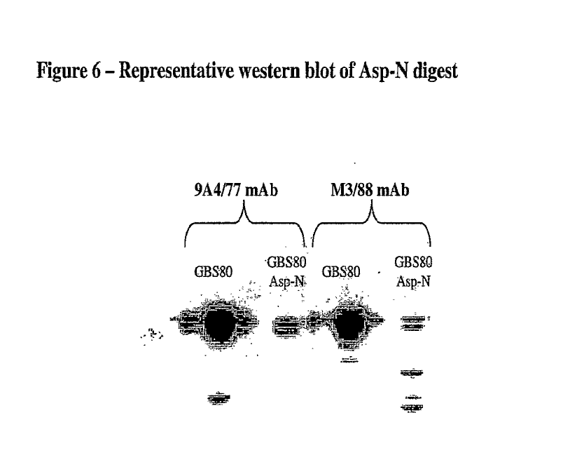

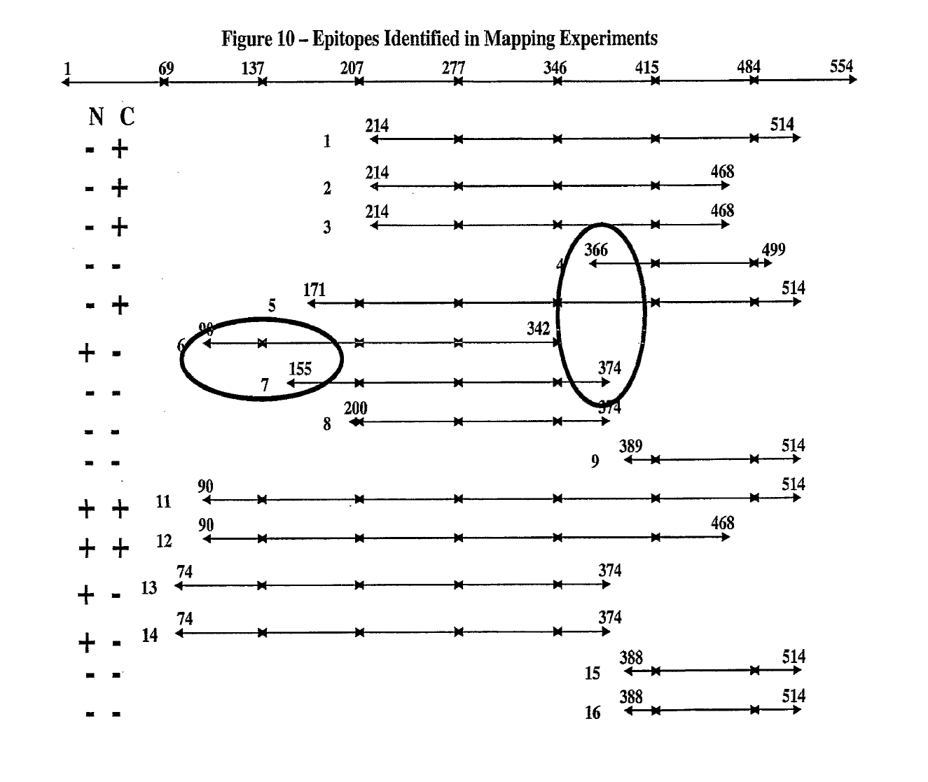

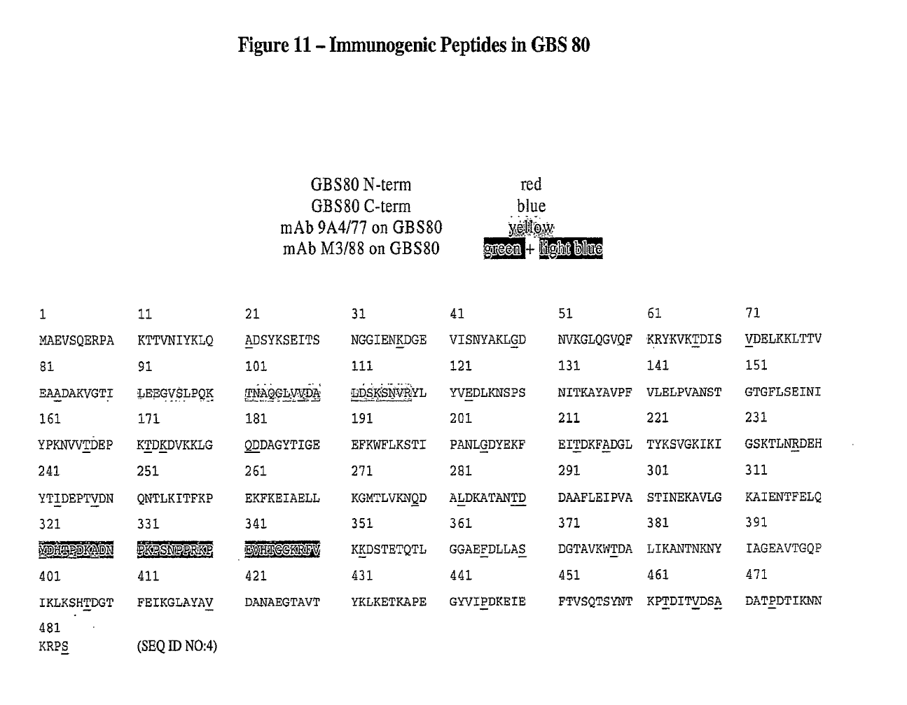

From the pattern of bands produced on western blot such as on FIGS. 6 and 8, the epitopes bound by the antibodies were identified. FIG. 10 summarizes the results of the western blots. The fifteen of the sixteen fragments from the SDS PAGE gel shown in FIG. 9 are displayed as horizontal bars. The pattern of bands observed in western blots is shown along the left with a (+) for each band observed and a (−) for each band missing. The N column corresponds to binding by the monoclonal antibody 9A4/77 and the C column corresponds to binding by the monoclonal antibody M3/88. The circle on the right indicates the epitope for 9A4/77 and the circle on the left indicates the epitope for M3/88. FIG. 11 shows the sequence of GBS 80 with the epitope for 9A4/77 highlighted in yellow and the epitope for M3/88 highlighted in light blue with the core in green.

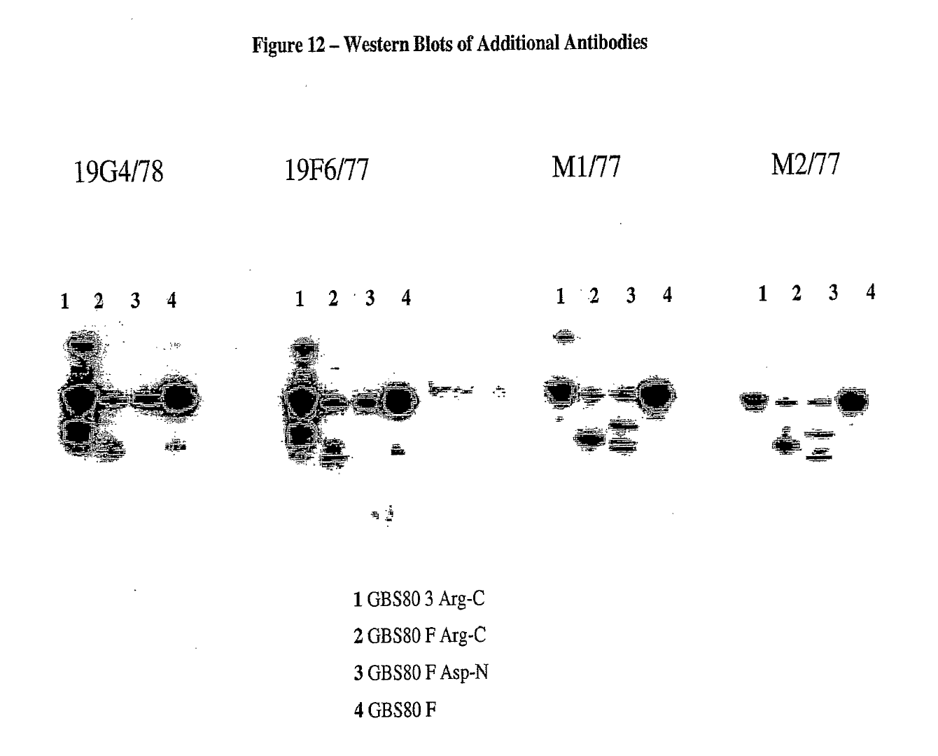

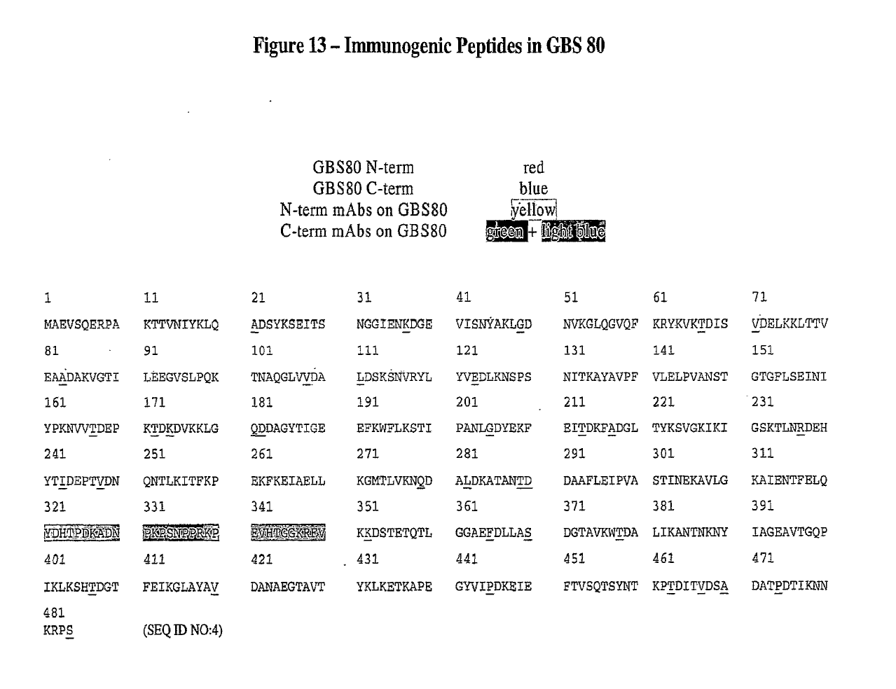

Additional epitope mapping with the other four monoclonal antibodies produced similar results. Representative western blots for the other four monoclonal antibodies are shown in FIG. 12. The results are summarized in FIG. 13, which shows the sequence of GBS 80 with the epitope for the N-terminal monoclonal antibodies (9A4/77, 19G4/78 and 19F6/77) highlighted in yellow and the epitope for M3/88 highlighted in light blue with the core in green. The C-terminal epitope is the same, while the N-terminal epitope is a bit more extensive than the epitope for 9A4/77 alone. The results of this Example 2 demonstrate that GBS 80 contains at least three immunogenic polypeptides corresponding to amino acids: 54-118, 38-118, and 321-350.

### Example 3

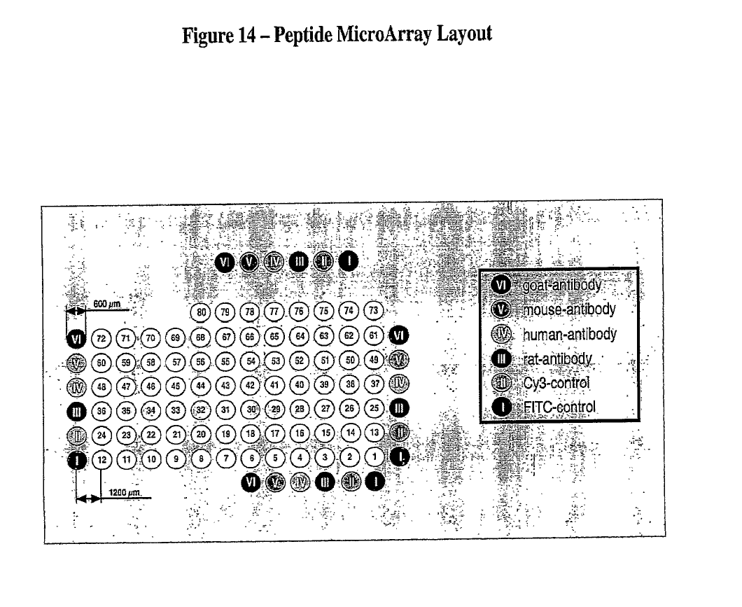

Additional epitope mapping was conducted to identify immunogenic polypeptides of the present invention using peptide arrays. A RepliTope™ peptide microarray (JPT Peptide Technologies) was procured which had a series of overlapping peptide fragments of GBS 80 affixed to it in triplicate. The peptide fragments listed in Table 3 were arranged on the microarray slide as shown in FIG. 14. The pattern shown in FIG. 14 was replicated three times on the microarray slide.

TABLE 3
Peptide sequences on the MicroArray

| Position on | | |
|---|---|---|
| the MicroArray | Peptide Sequence | SEQ ID NO: |
| 1 | DAAFLEIPVASTI | 13 |
| 2 | FLEIPVASTINEK | 14 |
| 3 | IPVASTINEKAVL | 15 |
| 4 | ASTINEKAVLGKA | 16 |
| 5 | INEKAVLGKAIEN | 17 |
| 6 | KAVLGKAIENTFE | 18 |
| 7 | LGKAIENTFELQY | 19 |
| 8 | AIENTFELQYDHT | 20 |
| 9 | NTFELQYDHTPDK | 21 |
| 10 | ELQYDHTPDKADN | 22 |
| 11 | YDHTPDKADNPKP | 23 |
| 12 | TPDKADNPKPSNP | 24 |
| 13 | KADNPKPSNPPRK | 25 |
| 14 | NPKPSNPPRKPEV | 26 |
| 15 | PSNPPRKPEVHTG | 27 |
| 16 | PPRKPEVHTGGKR | 28 |
| 17 | KPEVHTGGKRFVK | 29 |
| 18 | VHTGGKRFVKKDS | 30 |
| 19 | GGKRFVKKDSTET | 31 |
| 20 | RFVKKDSTETQTL | 32 |
| 21 | KKDSTETQTLGGA | 33 |
| 22 | STETQTLGGAEFD | 34 |
| 23 | TQTLGGAEFDLLA | 35 |
| 24 | LGGAEFDLLASDG | 36 |
| 25 | AEFDLLASDGTAV | 37 |
| 26 | DLLASDGTAVKWT | 38 |
| 27 | LASDGTAVKWTDA | 39 |
| 37 | MAEVSQERPAKTT | 40 |
| 38 | VSQERPAKTTVNI | 41 |
| 39 | ERPAKTTVNIYKL | 42 |
| 40 | AKTTVNIYKLQAD | 43 |
| 41 | TVNIYKLQADSYK | 44 |
| 42 | IYKLQADSYKSEI | 45 |
| 43 | LQADSYKSEITSN | 46 |
| 44 | DSYKSEITSNGGI | 47 |
| 45 | KSEITSNGGIENK | 48 |
| 46 | ITSNGGIENKDGE | 49 |
| 47 | NGGIENKDGEVIS | 50 |
| 48 | IENKDGEVISNYA | 51 |
| 49 | KDGEVISNYAKLG | 52 |
| 50 | EVISNYAKLGDNV | 53 |
| 51 | SNYAKLGDNVKGL | 54 |
| 52 | AKLGDNVKGLQGV | 55 |
| 53 | GDNVKGLQGVQFK | 56 |
| 54 | VKGLQGVQFKRYK | 57 |
| 55 | LQGVQFKRYKVKT | 58 |
| 56 | VQFKRYKVKTDIS | 59 |
| 57 | KRYKVKTDISVDE | 60 |
| 58 | KVKTDISVDELKK | 61 |
| 59 | TDISVDELKKLTT | 62 |
| 60 | SVDELKKLTTVEA | 63 |
| 61 | ELKKLTTVEAADA | 64 |
| 62 | KLTTVEAADAKVG | 65 |
| 63 | TVEAADAKVGTIL | 66 |
| 64 | AADAKVGTILEEG | 67 |
| 65 | AKVGTILEEGVSL | 68 |
| 66 | GTILEEGVSLPQK | 69 |
| 67 | LEEGVSLPQKTNA | 70 |
| 68 | GVSLPQKTNAQGL | 71 |
| 69 | LPQKTNAQGLVVD | 72 |
| 70 | KTNAQGLVVDALD | 73 |
| 71 | AQGLVVDALDSKS | 74 |
| 72 | LVVDALDSKSNVR | 75 |
| 73 | DALDSKSNVRYLY | 11 |
| 74 | DSKSNVRYLYVED | 76 |
| 75 | SNVRYLYVEDLKN | 12 |
| 76 | RYLYVEDLKNSPS | 77 |
| 77 | YVEDLKNSPSNIT | 78 |
| 78 | DLKNSPSNITKAY | 79 |
| 79 | NSPSNITKAYAVP | 80 |
| 80 | SPSNITKAYAVPF | 81 |

The peptide microarray slide was used according to the following procedure:
Treat the slide and the coverslip with a solution of 0.1 mg/ml polyvinylpyrrolidone overnight at 4° C.
Wash 2 times with H₂O for 5 minutes.
Incubate with monoclonal antibody 9A4/77 diluted 1:300 in PBS for 1 hour at room temperature.
Wash 4 times with PBS-Tween 0.1% for 5 minutes.
Wash 2 times with PBS for 5 minutes.
Incubate with anti-mouse antibody labeled with Cy5 diluted 1:300 in PBS for 1 hour at room temperature.
Wash 4 times with PBS-Tween 0.1% for 5 minutes.
Wash 3 times with H₂O for 5 minutes.
Dry the slide using a stream of nitrogen.
Perform fluorescence scans.

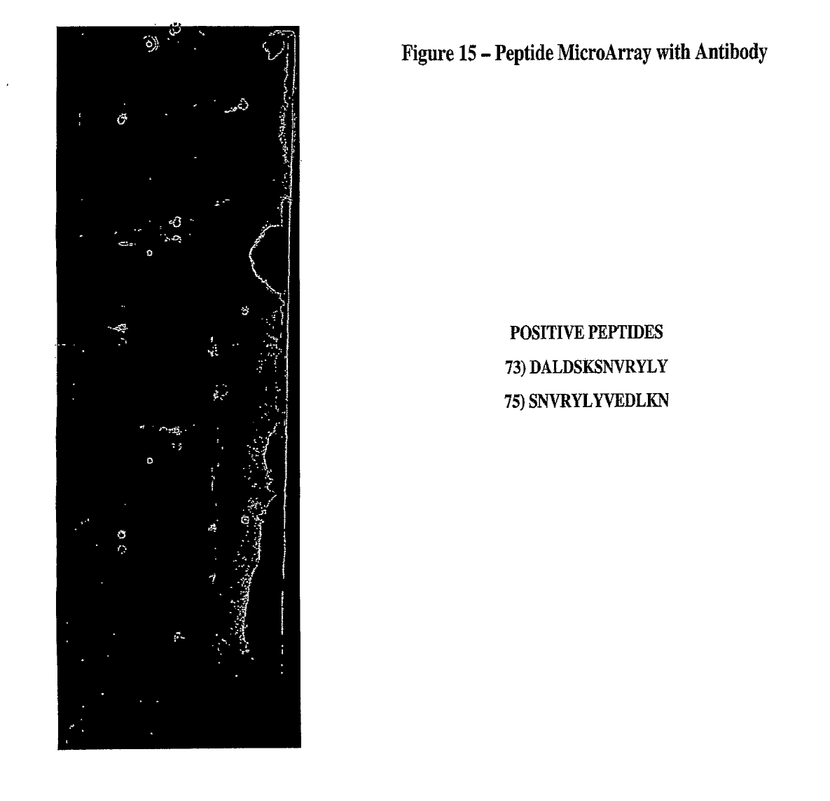

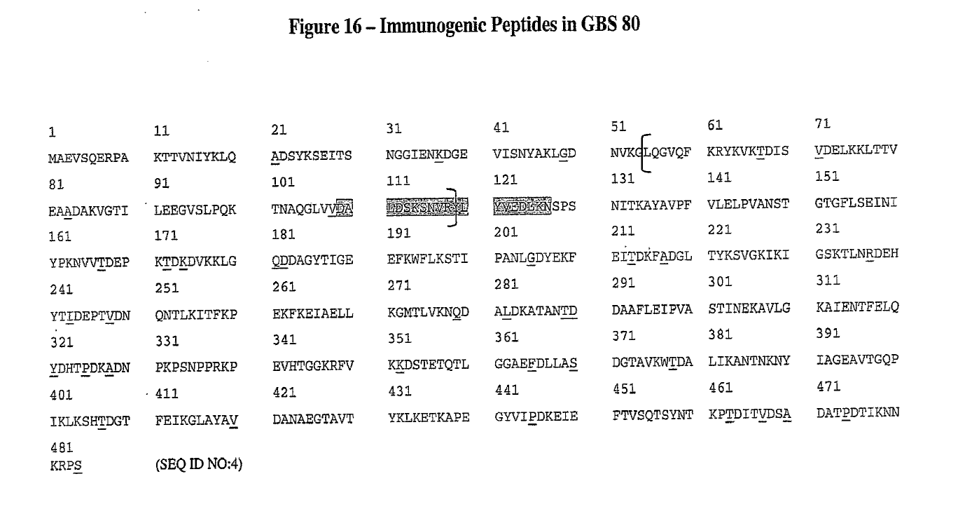

The image from the fluorescence scan is shown in FIG. 15. The control spots are indicated with a dashed circle. The GBS 80 immunogenic polypeptides are indicated with a solid circle. The monoclonal antibody 9A4/77 bound to two polypeptides: SEQ ID NO:11 and SEQ ID NO:13. FIG. 16 shows the position of SEQ ID NO:11 relative to the immunogenic polypeptide identified by western blotting (between the blue brackets).

### ADDITIONAL REFERENCES

### The Contents of which are Hereby Incorporated by Reference

1. Tettelin et al. (2002) Proc. Natl. Acad. Sci. USA, 10.1 073/pnas. 182380799.
2. International patent application WO02/34771.
3. Terpe et al., “Overview of tag protein fusions: from molecular and biochemical fundamentals to commercial systems”, Appl Microbiol Biotechnol (2003) 60:523-533.
4. WO99/27961.
5. WO02/074244.
6. WO02/064162.
7. WO03/028760.
8. Gennaro (2000) Remington: The Science and Practice of Pharmacy. 20th ed., ISBN: 0683306472.
9. Vaccine design: the subunit and adjuvant approach (1995) Powell & Newman. ISBN 0-306-44867-X.
10. WO00/23105.
11. WO00/07621.
12. Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.
13. Niikura et al., “Chimeric Recombinant Hepatitis E Virus-Like Particles as an Oral Vaccine Vehicle Presenting Foreign Epitopes”, Virology (2002) 293:273-280.
14. Lenz et al., “Papillomarivurs-Like Particles Induce Acute Activation of Dendritic Cells”, Journal of Immunology (2001) 5246-5355.
15. Pinto, et al., “Cellular Immune Responses to Human Papillomavirus (HPV)-16 L1 Healthy Volunteers Immunized with Recombinant HPV-16 L1 Virus Like Particles”, Journal of Infectious Diseases (2003)/88:327-338.
16. Gerber et al., “Human Papillomavirus Virus-Like Particles Are Efficient Oral Immunogens when Coadministered with Escherichia coli Heat-Labile Enterotoxin Mutant R192G or CpG”, Journal of Virology (2001) 75(10):4752-4760.
17. Gluck et al., “New Technology Platforms in the Development of Vaccines for the Future”, Vaccine (2002) 20:B10-B16.
18. Johnson et al. (1999) Bioorg Med Chem Lett 9:2273-2278
19. Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491.
20. Pajak, et al., “The Adjuvant OM-174 induces both the migration and maturation of murine dendritic cells in vivo”, Vaccine (2003) 21:836-842.
21. Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 239 3-2400.
22. Krieg, “CpG motifs: the active ingredient in bacterial extracts?”, Nature Medicine (2003) 9(7): 831-835.
23. McCluskie, et al., “Parenteral and mucosal prime-boost immunization strategies in mice with hepatitis B surface antigen and CpG DNA”, FEMS Immunology and Medical Microbiology (2002) 32:179 185.
24. Kandimalla, et al., “Toll-like receptor 9: modulation of recognition and cytokine induction by novel synthetic CpG DNAs”, Biochemical Society Transactions (2003) 31 (part 3): 654-658.
25. Blackwell, et al., “CpG-A-Induced Monocyte IFN-gamma-Inducible Protein-10 Production is Regulated by Plasmacytoid Dendritic Cell Derived IFN-alpha”, J. Immunol. (2003)170(8):4061 4068.
26. Krieg, “From A to Z on CpG”, TRENDS in Immunology (2002) 23(2): 64-65.
27. Kandimalla, et al., “Secondary structures in CpG oligonucleotides affect immunostimulatory activity”, BBRC (2003) 306:948-953.
28. Kandimalla, et al., “Toll-like receptor 9: modulation of recognition and cytokine induction by novel synthetic GpG DNAs”, Biochemical Society Transactions (2003) 31(part 3):664-658.
29. Bhagat et al., “CpG penta and hexadeoxyribonucleotides as potent immunomodulatory agents” BBRC (2003) 300:853-861.
30. Singh et al. (2001).1. Cont. Rele. 70:267-276.
31. WO99/27960.
32. WO99/52549.
33. WO01/21207.
34. WO01121152.
35. Andrianov et al., “Preparation of hydrogel microspheres by coacervation of aqueous polyphophazene solutions”, Biomaterials (1998) 19(1-3):109-115.
36. Payne et al., “Protein Release from Polyphosphazene Matrices”, Adv. Drug. Delivery Review (1998) 31(3):185-196.
37. Stanley, “Imiqulmod and the imidazoquinolones: mechanism of action and therapeutic potential” Clin Exp Dermatol (2002) 27(7):571-577.
38. Jones, “Resiquimod 3M”, Curr Opin Investig Drugs (2003) 4(2):214-218.
39. WO99/11241.
40. WO98/57659.
41. European patent applications 0835318, 0735898 and 0761231.
42. Ramsay et al. (2001) Lancet 357(9251): 195-196.
43. Lindberg (1999) Vaccine 17 Suppl 2:S28-36.
44. Buttery & Moxon (2000) J R Coll Physicians Lond 34:163-168.
45. Ahmad & Chapnick (1999) Infect Dis Clin North Am 13: 113 133, vii.
46. Goldblatt (1998) J. Med. Microbiol. 47:563-567.
47. European patent 0 477 508.
48. U.S. Pat. No. 5,306,492.
49. International patent application WO98/42721.
50. Conjugate Vaccines (eds. Cruse et al.) ISBN 3805549326, particularly vol. 10:48-114.
51. Hermanson (1996) Bioconjugate Techniques ISBN: 0123423368 or 012342335X.
52. Research Disclosure, 453077 (January 2002)
53. EP-A-0372501
54. EP-A-0378881
55. EP-A-0427347
56. WO93/17712
57. WO94/03208
58. WO98/58668
59. EP-A-0471177
60. WO00/56360
61. WO91/01146
62. WO00/61761
63. WO01/72337
64. Robinson & Torres (1997) Seminars in Immunology 9:271-283.
65. Donnelly et al. (1997) Annu Rev Immunol 15:617-648.
66. Scott-Taylor & Dalgleish (2000) Expert Opin Investig Drugs 9:471-480.
67. Apostolopoulos & Plebanski (2000) Curr Opin Mol. Ther. 2:441-447.
68. Ilan (1999) Curr Opin Mol. Ther. 1:116-120.
69. Dubensky et al. (2000) Mol. Med. 6:723-732.
70. Robinson & Pertmer (2000) Adv Virus Res 55: 1-74.
71. Donnelly et al. (2000) Am J Respir Crit. Care Med 162(4 Pt 2):S190-193.
72. Davis (1999) Mt. Sinai J. Med. 66:84-90.
73. Current Protocols in Molecular Biology (F. M. Ausubel et al., eds., 1987) Supplement 30.
74. Smith & Waterman (1981) Rev. Appl. Math. 2: 482-489.
75. U.S. Pat. No. 6,372,223.
76. WO00/15251.
77. WO01/22992.
78. Hehme et al. (2004) Virus Res. 103(1-2):163-71.
79. U.S. Pat. No. 6,355,271.
80. WO00/23105.
81. Vaccine Design: The Subunit and Adjuvant Approach (eds. Powell & Newman) Plenum Press 1995 (ISBN 0-306-44867-X).
82. U.S. Pat. No. 5,057,540.
83. WO05/02620.
84. WO96/33739.
85. EP-A-0109942.
86. U.S. Pat. No. 4,578,269.
87. WO96/11711.
88. U.S. Pat. No. 6,352,697.
89. WO00/07621 and U.S. Pat. No. 6,506,386.
90. WO04/04762.
91. Barr et al. (1998) Advanced Drug Delivery Reviews 32:247-271.
92. Sjolanderet et al (1998) Advanced Drug Delivery Reviews 32:321-338.
93. Pizza et al. (2000) Int J Med Microbiol 290:455-461.
94. WO95/17211.
95. WO98/42375.
96. Singh et al. (2001) J Cont Release 70:267-276.
97. WO99/27960.
98. U.S. Pat. No. 6,090,406.
99. U.S. Pat. No. 5,916,588.
100. EP-A-0626169.
101. WO99/52549.
102. WO01/21207.
103. WO01/21152.
104. WO02/72012.
105. Signorelli and Hadden (2003) Int Immunopharmacol 3(8):1177-86.
106. WO04/64715.
107. Cooper (1995) Pharm Biotechnol 6:559-80.
108. Vaccine Adjuvants: Preparation Methods and Research Protocols (Volume 42 of Methods in Molecular Methods series). ISBN: 1-59259-083-7. Ed. O'Hagan.
109. WO05/89837.
110. U.S. Pat. No. 6,692,468.
111. WO00/07647.
112. WO99/17820.
113. U.S. Pat. No. 5,971,953.
114. U.S. Pat. No. 4,060,082.
115. EP-A-0520618.
116. WO98/01174.
117. WO90/14837.
118. Podda and Del Giudice (2003) Expert Rev Vaccines 2:197-203.
119. Podda (2001) Vacccine 19:2673-2680.
120. Allison and Byars (1992) Res Immunol 143:519-25.
121. Hariharan et al. (1995) Cancer Res 55:3486-9.
122. WO95/11700.
123. U.S. Pat. No. 6,080,725.
124. WO05/097181.
125. Han et al. (2005) Impact of Vitamin Eon Immune Function and Infectious Diseases in the Aged at Nutrition, Immune functions and Health EuroConference, Paris, 9-10 Jun. 2005.
126. U.S. Pat. No. 6,630,161.
127. WO02/097072.
128. Hayden et al. (1998) J Clin Invest 101(3):643-9.
129. Tassignon et al. (2005) J Immunol Meth 305:188-98.
130. Myers et al. (1990) pages 145-156 of Cellular and molecular aspects of endotoxin reactions.
131. Ulrich (2000) Chapter 16 (pages 273-282) of reference 108.
132. Johnson et al. (1999) J Med Chem 42:4640-9_{—}
133. Baldrick et al. (2002) Regulatory Toxicol Pharmacol 35:398-413.
134. U.S. Pat. No. 4,680,338.
135. U.S. Pat. No. 4,988,815.
136. WO92/15582.
137. Stanley (2002) Clin Exp Dermatol 27:571-577.
138. Wu et al. (2004) Antiviral Res. 64(2):79-83.
139. Vasilakos et al. (2000) Cell Immunol. 204(464-74.
140. U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
141. Jones (2003) Curr Opin Investig Drugs 4:214-218.
142. WO2004/060308.
143. WO2004/064759.
144. U.S. Pat. No. 6,924,271.
145. U.S. Patent App. No. 2005/0070556.
146. U.S. Pat. No. 5,658,731.
147. U.S. Pat. No. 5,011,828.
148. WO2004/87153.
149. U.S. Pat. No. 6,605,617.
150. WO02/18383.
151. WO2004/018455.
152. WO03/082272.
153. PCT/US2005/022769.
154. Johnson et al. (1999) Bioorg Med Chem Lett 9:2273-2278.
155. Evans et al. (2003) Expert Rev Vaccines 2:219-229.
156. Andrianov et al. (1998) Biomaterials 19:109-115.
157. Payne et al. (1998) Adv Drug Delivery Review 31:185-196.
158. Thompson et al. (2003) Methods in Molecular Medicine 94:255-266.
159. Kandimalla et al. (2003) Nucleic Acids Research 31:2393-2400.
160. WO02/26757.
161. WO99/62923.
162. Krieg (2003) Nature Medicine 9:831-835.
163. McCluskie et al. (2002) FEMS Immunology and Medical Microbiology 32:179-185.
164. WO98/40100.
165. U.S. Pat. No. 6,207,646.
166. U.S. Pat. No. 6,239,116.
167. U.S. Pat. No. 6,429,199.
168. Kandimalla et al. (2003) Biochemical Society Transactions 31 (part 3):654-658.
169. Blackwell et al. (2003) J Immunol 170:4061-4068.
170. Krieg (2002) Trends Immunol 23:64-65.
171. WO01/95935.
172. Kandimalla et al. (2003) BBRC 306:948-953.
173. Bhagat et al. (2003) BBRC 300:853-861.
174. WO03/035836.
175. WO01/22972.
176. Thompson et al. (2005) J Leukoc Biol 78: ‘The low-toxicity versions of LPS, MPLO adjuvant and RC529, are efficient adjuvants for CD4+ T cells’.
177. UK patent application GB A 2220211.
178. WO 94/21292.
179. WO94/00153.
180. WO95/17210.
181. WO96/26741.
182. WO93/19780.
183. WO03/011223.
184. Meraldi et al. (2003) Vaccine 21:2485-2491.
185. Pajak et al. (2003) Vaccine 21:836-842.
186. U.S. Pat. No. 6,586,409.
187. Wong et al. (2003) J Clin Pharmacol 43(7):735-42.
188: U.S. Pat. App. No. 2005/0215517.

1. An isolated polypeptide comprising the amino acid sequence SEQ ID NO:5, provided that the isolated polypeptide does not consist of the amino acid sequence SEQ ID NO:2.

2. The isolated polypeptide of claim 1 which is a recombinant polypeptide.

3. A composition comprising:
an isolated polypeptide comprising the amino acid sequence SEQ ID NO:5, provided that the isolated polypeptide does not consist of the amino acid sequence SEQ ID NO:2; and
a pharmaceutically acceptable carrier.

4. The composition of claim 3, wherein the isolated polypeptide is a recombinant polypeptide.

5. The composition of claim 3, wherein the composition is a vaccine.

6. The composition of claim 5, wherein the isolated polypeptide is a recombinant polypeptide.

7. The composition of claim 3, further comprising an adjuvant.

8. The composition of claim 7, wherein the isolated polypeptide is a recombinant polypeptide.

9. A method of inducing an immune response against S. agalactiae, comprising administering to an individual an effective amount of a polypeptide comprising the amino acid sequence SEQ ID NO:5, provided that the isolated polypeptide does not consist of the amino acid sequence SEQ ID NO:2.

10. The method of claim 9, wherein the isolated polypeptide is a recombinant polypeptide.

11. The method of claim 9, wherein the isolated polypeptide is provided by a composition comprising a pharmaceutically acceptable carrier.

12. The method of claim 11, wherein the isolated polypeptide is a recombinant polypeptide.

13. The method of claim 9, wherein the composition further comprises an adjuvant.

14. The method of claim 13, wherein the isolated polypeptide is a recombinant polypeptide.

15. The method of claim 9, wherein the composition is a vaccine.

16. The method of claim 15, wherein the isolated polypeptide is a recombinant polypeptide.
